(19) United States
(12) Patent Application Publication
(10) Pub. No.: US 2026/0042889 A1
(43) Pub. Date: Feb. 12, 2026

(54) SYSTEMS AND METHODS FOR SELECTIVE SHAPE TRANSFORMATION OF 3D-PRINTED MATERIALS

(71) Applicants: University of Washington, Seattle, WA (US); ASOCIACIÓN CENTRO DE INVESTIGACIÓN COOPERATIVA EN BIOMATERIALES - CIC BIOMAGUNE, Guipúzcoa, Donostia-San Sebastián (ES); UNIVERSITY OF THE BASQUE COUNTRY, UPV/EHU, Leioa, Bizkaia (ES)

(72) Inventors: Alshakim Nelson, Seattle, WA (US); Eva Sánchez Rexach, Seattle, WA (US); Naroa Sadaba, Seattle, WA (US); Siwei Yu, Seattle, WA (US); Haritz Sardón Muguruza, Leioa, Bizkaia (ES); Dorleta Jiménez de Aberasturi, Guipúzcoa (ES); Luis Manuel Liz-Marzan, Guipúzcoa (ES)

(73) Assignees: University of Washington, Seattle, WA (US); ASOCIACIÓN CENTRO DE INVESTIGACIÓN COOPERATIVA EN BIOMATERIALES - CIC BIOMAGUNE, Guipúzcoa, Donostia-San Sebastián (ES); UNIVERSITY OF THE BASQUE COUNTRY, UPV/EHU, Leioa, Bizkaia (ES)

(21) Appl. No.: 19/101,233

(22) PCT Filed: Aug. 4, 2023
(86) PCT No.: PCT/US2023/029568

§ 371 Date: Feb. 4, 2025

Related U.S. Application Data
(60) Provisional application No. 63/395,645, filed on Aug. 5, 2022.

(51) Int. Cl. C08J 3/075; A61L 31/04; A61L 31/14; B33Y 70/00; B33Y 80/00

(52) U.S. Cl. CPC C08J 3/075; A61L 31/047; A61L 31/145; A61L 2400/12; A61L 2400/16; B33Y 70/00; B33Y 80/00; C08J 2389/00

(57) ABSTRACT

Shape-restoring materials, as well as techniques for generating shape-restoring materials, are described. An example method includes generating a construct by exposing, to UV-visible light, a resin comprising a globular protein, a water-soluble co-monomer, light-to-heat converting nanoparticles, water, and a photoinitiator. At least a portion of the water is removed from the construct. The construct is converted from a first shape to a second shape by applying a force to the construct. The construct is reverted to the first shape in response to being exposed to NIR light, due to the absorption of the NIR light by the nanoparticles.

### CROSS-REFERENCE TO RELATED APPLICATION(S)

This application is a U.S. National Phase Application which claims the priority of International Application No. PCT/US2023/029568, filed Aug. 4, 2023, which claims the priority of U.S. Provisional App. No. 63/395,645, filed on Aug. 5, 2022, the entire contents of each of which are incorporated by reference herein in their entirety.

### STATEMENT REGARDING FEDERALLY SPONSORED RESEARCH OR DEVELOPMENT

This invention was made with government support under Grant No. W81XWH-21-1-0167, awarded by the Congressionally Directed Medical Research Programs and Grant No. 787510, awarded by the European Research Council. The government has certain rights in the invention.

### TECHNICAL FIELD

This application relates to shape-restoring materials (e.g., shape-restoring bioplastics), as well as methods and systems for generating shape-restoring materials.

### BACKGROUND

Shape memory materials have a range of applications that include intelligent robotic, medical deceives, origami structure, sports, and fashion (S. Joshi, et al., A.s.s, Appl. Mater. Today 2020, 18, 100490; J. Zhang, et al., Adv. Mater. Technol. 2022, 7, 2101568; M. C. Biswas, et al., Adv. Funct. Mater. 2021, 31, 2100257). The combination of shape memory materials with additive manufacturing has led to the field of four-dimensional (4D) printing, which is the three-dimensional (3D) printing of objects that can change chemically or physically in response to an external stimulus, (A. Cortés, et al., Adv. Funct. Mater. 2021, 31, 2106774) such as water (Z. Fang, et al., G. Chen, ACS Appl. Mater. Interfaces 2017, 9, 5495), light (M. Herath, et al., Eur. Polym. J. 2020, 136, 109912), temperature (X. Xiao, et al., Macromolecules 2015, 48, 3582), or magnetic/electric field (Q. Ze, et al., Adv. Mater. 2020, 32, 1906657). In this way, printed objects are no longer static objects and become programmable dynamic objects, which are able to change shape in response to an exogenous input.

Among shape memory materials, shape memory polymers (SMP) have been well used and studied as they have great recovery behavior and tunability (Y. Wang, et al., Adv. Funct. Mater. n.d., n/a, 2210614). Some well-established SMPs are polylactic acid (PLA) (J. Xu and J. Song, in Shape Mem. Polym. Biomed. Appo. (Ed.: L. Yahia), Woodhead Publishing, 2015, pp. 197-217), poly(e-caprolactone) diacrylate (PCL-DA) (F. O. Beltran, et al., ACS Biomater. Sci. Eng. 2021, 7, 1631), and polynorbornene (C. Liu, et al., J. Mater. Chem. 2007, 17, 1543).

Light activation of SMPs can be direct or indirect (Y. Wang, et al., Mv. Mater. Technol. 2022, 7, 2101058). In direct light activation, the polymer photochemically responds to light, such as cis-trans isomerization (M. Herath, et al., Eur. Polym. J. 2020, 136, 109912). Indirect light activation is dependent upon the conversion of light into a second source of energy input such as heat (K. Jiang, et al., J. Phys. Chem. C 2013, 117, 27073). The thermal energy locally raises the temperature of the material above the glass transition temperature (Tg) to facilitate the shape recovery (Y. Wang, et al., Mv. Mater. Technol. 2022, 7, 2101058). Carbon nanotube (S. S. Mahapatra, et al., Polymer 2019, 160, 204), graphene (J. Loomis, et al., Sci. Rep. 2013, 3, 1900), and metal nanoparticles and nanorods (Y. Wang, et al., Adv. Funct. Mater. n.d., n/a; Y. Wang, et al., Adv. Mater. Technol. 2022, 7, 2101058. 2210614; H. Zhang and Y. Zhao, ACS Appl. Mater. Interfaces 2013, 5, 13069; N. Yenpech, et al., Polymer 2019, 182, 121792) are used as photothermal converters. Photothermal activation has significant advantage over the direct light or heat activation as it can achieve more homogenous heat distribution (faster recovery time), remote and selective control of the recovery process (A. Cortés, et al., Mv. Funct. Mater. 2021, 31, 2106774).

Stereolithographic apparatus (SLA) 3D printing is a printing method that has a fast printing rate, high resolution, and good reproducibility (W. Li, et al., Mv. Healthc. Mater. 2020, 9, 2000156; E. M. Wits, et al., Polym. Chem. 2019, 10, 1442; F. P. W. Melchels, et al., Biomaterials 2010, 31, 6121). Despise the recent development of shape memory materials, there is a need for bio-sourced or protein-based materials that fulfill both the requirements of SLA printing and have shape memory behavior. SLA-printable resins have a viscosity between 0.2 to 10 Pascal second (Pa*s), which enables reflow in the SLA tray for printing subsequent layers (E. Sanchez-Rexach, et al., Chem. Mater. 2020, 32, 7105). There is also a need for resins with a relatively fast rate of photocuring at a chosen wavelength.

Acrylate and methacrylate functionalities can be used to make proteins photocurable. For example, silk fibroin methacrylate (Sil-MA) (S. For. H. For. Kim, et al., Nat. For. Commun. 2018, 9, 1620; A. For. Reizabal, et al., Adv. For. Funct. For. Mater. 2023, 33, 2210764; A. For. Bucciarelli, et al., Gels 2022, 8, 833), gelatin methacrylate (Gel-MA) (X. For. Zhou, et al., ACS Appo. For. Mater. For. Interfaces 2016, 8, 44), and methacrylate bovine serum albumin (MA-BSA) (E. For. Sanchez-Rexach, et al., ACS Appo. For. Mater. For. Interfaces 2021, 13, 19193; G. For. Ferracci, et al., ACS Appl. For. Bio Mater. 2020, 3, 920; P. For. T. For. Smith, et al., Biomacromolecules 2020, 21, 484) have been explored.

### BRIEF DESCRIPTION OF THE DRAWINGS

FIG. 1 illustrates an example environment for generating shape-restoring materials.

FIGS. 2A to 2C illustrate examples of various types of nanoparticles that can be used in various types of shape-restoring materials described herein. FIG. 2A illustrates an example of a nanorod having a certain width and length. FIG. 2B illustrates an example of a nanostar with a certain arm length. FIG. 2C illustrates an example of a triangle with a certain edge length.

FIGS. 3A to 3C illustrate an example of the use of a stent that includes a shape-restoring material.

FIGS. 4A and 4B illustrate an example of a construct that can be selectively bent and straightened using a shape-restoring material.

FIG. 5 illustrates an example process for utilizing a shape-restoring material.

FIGS. 6A to 6D illustrate examples of viscosity of resin determined using a rheometer. FIG. 6A shows a viscosity vs shear rate curve for an example bovine serum albumin (BSA) 3:1 resin with 0-0.002% of gold nanorods (AuNRs). FIG. 6B illustrates a viscosity vs shear rate curve for an example BSA 2:1 resin with 0-0.002% of AuNRs. FIG. 6C illustrates a viscosity vs shear rate curve for an example methacrylated BSA (MABSA) 3:1 resin with 0-0.002% of AuNRs. FIG. 6D illustrates a viscosity vs shear rate curve for an example MABSA 2:1 resin with 0-0.002% of AuNRs.

FIGS. 7A to 7D illustrate an example rate of photocuring of resin determined using a rheometer. FIG. 7A illustrates photorehometry for an example BSA 3:1 resin with 0-0.002% of AuNRs. FIG. 7B illustrates photorehometry for an example BSA 2:1 resin with 0-0.002% of AuNRs. FIG. 7C illustrates photorehometry for an example MABSA 3:1 resin with 0-0.002% of AuNRs. FIG. 7D illustrates photorehometry for an example MABSA 2:1 resin with 0-0.002% of AuNRs.

FIG. 8 illustrates example compressive stress vs strain curves of 3D printed BSA 2:1 bioplastics with different concentrations of AuNRs.

FIGS. 9A and 9B illustrate example compressive stress vs strain curves of 3D printed bioplastics. FIG. 9A illustrates results of an example MABSA 3:1 bioplastic with different concentrations of AuNRs. FIG. 9B illustrates results of an example MABSA 3:1 0.00375% bioplastic compared with its laser-recovered bioplastic.

FIGS. 10A to 10C illustrate thermal curves of examples of the 3D printed bioplastic recovery process under 3-minute irradiation of near infrared (NIR) laser (808 nm). FIG. 10A shows a time vs temperature curve for an example of BSA 3:1 0-0.005% AuNRs. FIG. 10B shows a time vs temperature curve for an example of MABSA 3:1 0-0.005% AuNRs. FIG. 10C shows a time vs temperature curve for an example of MABSA 2:1 0-0.005% AuNRs.

FIG. 11A is a schematic illustration of SLA 3D printing of an example AuNRs incorporated BSA or MA-BSA based resin and the molecular level structures of the bioplastic. FIG. 11B illustrates the UV-visible-near infrared (UV-VIS-NIR) spectrum of the synthesized gold nanorods used in this example, with an insert showing a transmission electron microscopy (TEM) image of PEG-coated AuNRs in water (Scale bar, 50 nm). FIG. 11C illustrates a general scheme for the workflow utilized in this example.

FIG. 12A illustrates differential scanning calorimetry (DSC) curves of 0.0035% example AuNRs bioplastics. FIG. 12B illustrates the UV-VIS-NIR spectrum for an example 3D-printed BSA 3:1 0.00375% film. FIG. 12C illustrates a temperature vs irradiation time graph for four different example formulated bioplastics with 0.0035% AuNRs. FIG. 12D illustrates a diagram for original, compressed, and laser recovered shape (Disk) for 0-0.002% AuNRs in examples of a BSA 2:1 formulated bioplastic.

FIGS. 13A to 13D illustrate compressive stress vs strain curves of example 3D printed bioplastics using BSA 3:1 bioplastics with 0-0.005% AuNRs (FIG. 13A), MABSA 3:1 bioplastic with 0-0.005% AuNRs (FIG. 13B), control samples (0% AuNRs) of 4 different formulated bioplastics (FIG. 13C), and BSA 3:1 0.00375% bioplastics compared with its laser-recovered bioplastic (FIG. 13D).

FIG. 14A illustrates an example of shape recovery of BSA 3:1 0.00375% 3D printed bioplastic ball under the pork gelatin skin. FIG. 14B illustrates an example of selective shape recovery of a folded 3D-printed BSA 3:1 four-arms flower.

### DETAILED DESCRIPTION

Various implementations described herein relate to shape-restoring materials, as well as methods and systems for generating and manipulating shape-restoring materials. Various resins including a globular protein, a co-monomer, and nanoparticles can be generated. Constructs can be generated by polymerizing the globular protein and the co-monomer in the resins, such as with the use of a photoinitiator. For instance, various photocurable resins described herein are suitable for stereolithographic printing. In some cases, the constructs are further dried.

The constructs can be generated in first shapes. Subsequently, the constructs can be reshaped into second shapes. For instance, the constructs can be reshaped by applying one or more forces to the constructs. In some cases, the second shapes are smaller than the first shapes, which enables the second shapes to be inserted into narrow openings that the first shapes would be unable to navigate. In various cases, the constructs can revert back into their first shapes in the presence of heat. In various examples described herein, the nanoparticles are configured to generate heat by absorbing light. Accordingly, the constructs may be restored to the first shapes by being exposed to the light that is converted to heat by the nanoparticles.

In particular examples, constructs described herein can be used for biomedical applications, such as within implantable apparatuses and devices. In some examples described herein, shape-restoring constructs can be generated using biocompatible materials, such as using biocompatible globular proteins, co-monomers, and light-to-heat converting nanoparticles. Further, the shape-restoring characteristics of various constructs described herein can enhance implantation procedures. For instance, an implantable device including an example shape-restoring construct can be inserted through a narrow port, incision, lumen, or other opening in the body of a subject while the construct is in a compressed state. Subsequently, the implantable device may expand, unfold, or otherwise change shape when the example shape-restoring construct is exposed to light that is absorbed by the nanoparticles in the construct. In some implementations, the nanoparticles are configured to convert NIR light into heat. Because NIR light can penetrate biological tissues (e.g., including blood, soft tissues, organs, etc.), implementations of the shape-restoring construct can be remotely reshaped by transmitting the NIR light through at least a portion of a body of a subject in which the implantable device is disposed. For instance, the shape of the construct can be adjusted after implantation in order to conform to an incision, organ, or other biological structure.

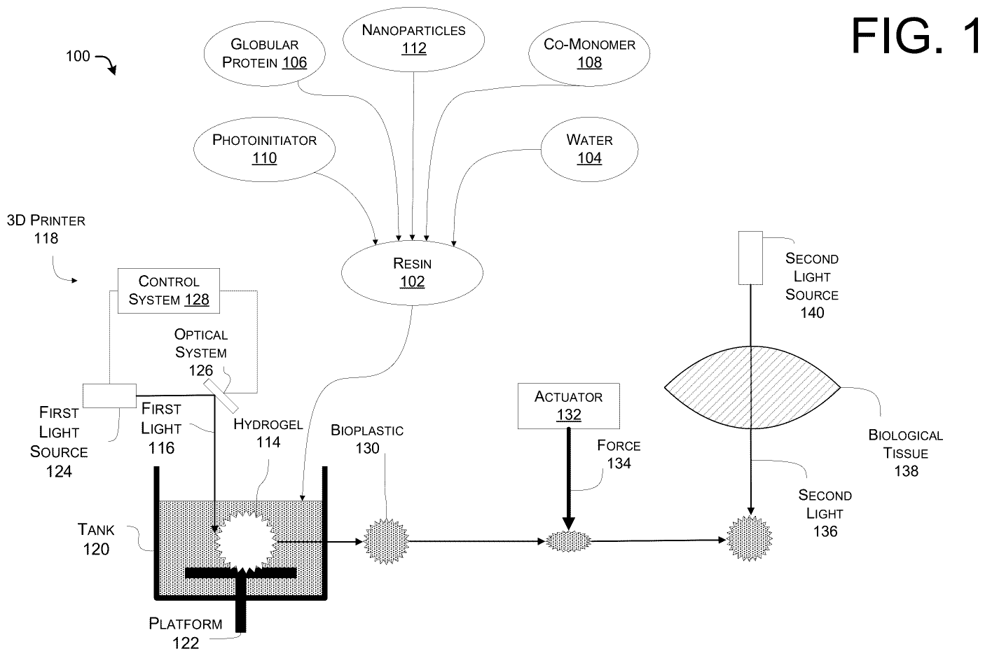

FIG. 1 illustrates an example environment 100 for generating shape-restoring materials. A resin 102 may be an aqueous solution that includes components configured to polymerize at a predetermined condition. In various implementations, the resin 102 includes various components, including water 104, a globular protein 106, and a co-monomer 108.

The globular protein 106 is soluble in the water 104. In various implementations, the globular protein 106 includes at least one polypeptide chain that is folded into a three-dimensional (3D) structure due to noncovalent interactions and disulfide bonds. In various examples, the globular protein 106 includes at least one of a serum albumin (e.g., bovine serum albumin (BSA)), pepsin, hemoglobin, lysozyme, lactoglobulin, pea protein, de novo protein, or soy protein. In some cases, the globular protein 106 includes one or more methacrylate or acrylate groups. Examples of the globular protein 106 include a methacrylated enzyme, methacrylated legume protein, methacrylated lysozyme, methacrylated lactoglobulin, methacrylated hemoglobin, methacrylated pepsin, or methacrylated serum albumin, acrylated enzyme, acrylated legume protein, acrylated lysozyme, acrylated lactoglobulin, acrylated hemoglobin, acrylated pepsin, or acrylated serum albumin. For instance, the globular protein 106 includes methacrylated bovine serum albumin (MABSA). In various implementations, the globular protein 106 is generated by exposing a non-methacrylated protein to a methacrylation reactant. For instance, the methacrylation reactant generates one or more methacrylate groups in the globular protein 106 by causing the non-methacrylated protein to undergo an amidation reaction and/or Michael addition reaction. Examples of methacrylation reactants include methacrylic anhydride and methacryloyl chloride.

Michael addition reactions involve the nucleophilic addition of a nucleophile to an α,β-unsaturated carbonyl compound containing an electron withdrawing group. Acrylated globular proteins are made using Michael addition reactions with compounds that have two or more acrylates. Examples of acrylation reactants include ethylene glycol diacrylate and polyethylene glycol diacrylate.

The co-monomer 108 is also soluble in water 104. In various cases, the co-monomer 108 includes an acrylate. For instance, the co-monomer 108 includes hydroxyethyl acrylate (HEA), acrylamide (AAm), polyethylene glycol diacrylate (PEG-DA), or a combination thereof. Other examples of acrylates include methacrylate, methyl acrylate, ethyl acrylate, acrylic anhydride, propargyl acrylate, allyl acrylate, polyethylene glycol monomethyl ether acrylate, butyl acrylate, and acrylic acid, 2-acrylamido-2-methylpropane sulfonic acid, and sodium 2-acrylamido-2-methylpropane sulfonate. In various examples, 5% to 95% of the resin 102, by weight, is the co-monomer 108.

According to some examples, the amount of the co-monomer 108 in the resin 102 is minimized, to reduce degradation of constructs generated from the resin 102. In various cases, the amount of the co-monomer 108 in the resin 102 is less, by weight, than the amount of the globular protein 106 in the resin 102. In some cases, a ratio (by weight) of the globular protein 106 to the co-monomer 108 in the resin is 1:1, 2:1, 3:1, 4:1, or 5:1.

In addition, the resin 102 may include a photoinitiator 110. The photoinitiator 110, according to various implementations, is water soluble. The photoinitiator 110, for instance, induces radical photopolymerization and/or cationic photopolymerization of the globular protein 106 and the co-monomer 108 when activated. In some examples, the photoinitiator 110 includes an initiator and a co-initiator. According to various implementations, the photoinitiator 110 includes an alpha hydroxyketone or derivative (e.g., 2-hydroxy-1-(4-(2-hydroxyethoxy) phenyl]-2-methyl-1-propanone (Irgacure 2959), 1-hydroxy-cyclohexyl-phenylketone (Irgacure 184), 2-benzyl-2-dimethylamino-1-(4-morpholinophenyl)-1-butanone (Irgacure 369), 2-methyl-4′-(methylthio-2-morpholinopropiophenone (Irgacure 907), 2-methyl-4-(methylthio)-2-morpholinopropiophenone (Irgacure 907), or sodium 4-(2-(4-morpholino)benzoyl-2-dimethylamino]butylbenzenesulphone (MBS)), a phosphine derivative (e.g., diphenyl(2,4,6-trimethylbenzoyl)phosphine oxide (TPO) or lithium phenyl-2,4,6-trimethylbenzoylphosphinate (LAP)), an azo-initiator (e.g., 2,2′-azobis (2-methyl-N-(2-hydroxyethyl) promionamide](VA-086)), eosin-Y and a co-initiator (e.g., an amine, such as triethanolamine or ethylamine), carboxylated camphorquinone and a co-initiator (e.g., an amine, such as triethylenamine and ethyl-4-N,N-dimethylaminobenzoate), riboflavin and a co-initiator (e.g., an amine, such as triethylamine), erythrosine and a co-initiator, or rose Bengal and a co-initiator. According to some cases, the photoinitiator 110 includes lithium phenyl-2,4,6-trimethylbenzoylphosphinate or 2-hydroxy-2-methylpropiophenone. In particular examples, the photoinitiator 110 is activated by light having a wavelength of 405 nanometers (nm) and includes a ruthenium complex (such as tris(2,2′-bipyridyl)dichlororuthenium(II) hexahydrate (Ru(bpy)₃) and a radical generator.

According to various implementations, the resin 102 further includes nanoparticles 112. As used herein, the terms “nanoparticle,” “nanostructure,” and their equivalents, may refer to a particle that has at least one dimension in a range of 1 to 100 nm. For example, a diameter, width, length, or combinations thereof, of the particle may be in a range of 1 to 100 nm. In various cases, the nanoparticles 112 are configured to convert energy from electromagnetic waves into heat. That is, the nanoparticles 112 may be light-to-heat converting nanoparticles. In various instances, the nanoparticles 112 may absorb near-infrared (NIR) light. As used herein, the terms “near infrared,” “NIR,” and their equivalents, may refer to light having a wavelength in a range of 750 nm to 2500 nm. For example, the nanoparticles 112 may convert light having a wavelength in a range of 800 to 900 nm into heat. In various cases, biological tissues (e.g., of mammals, such as humans) have limited absorption and scattering of NIR light. For instance, NIR light can be substantially transmitted through blood, adipose tissue, muscle, skin (e.g., including melanin), tendons, organs, and other types of biological tissues.

The nanoparticles 112 may have one or more shapes. In various cases, the nanoparticles 112 include at least one shape that creates a plasmon resonance at a wavelength of NIR light. As used herein, the terms “surface plasmon resonance,” “plasmon resonance,” “SPR,” “localized surface plasmon resonance,” “LSPR,” and their equivalents, may refer to a phenomenon that occurs when an electromagnetic stimulus (e.g., light) excites electrons on a surface (e.g., a metal surface) such that they travel parallel to the surface. In various cases, surface plasmon resonance occurs when the nanoparticles 112 are irradiated with excitation light having a particular range of wavelengths, and does not occur (e.g., does not significantly occur) when the nanoparticles are irradiated with light outside of the particular range of wavelengths. As used herein, the terms “surface plasmon resonance band,” “plasmon band,” “SPR band,” “LSPR band,” and their equivalents, may refer to a range of wavelengths of excitation light that trigger surface plasmon resonance of a material and/or a solution including the material. In some implementations, a surface plasmon resonance band may be defined based on an absorption spectrum of the material and/or the solution including the material. In various cases, an absorption spectrum is defined as an amount of absorption of the material and/or the solution including the material with respect to different wavelengths of excitation light. For example, the material may have a peak absorption at a particular wavelength of excitation light. In some cases, the surface plasmon resonance band includes a range of wavelengths of light that have greater than a threshold percentage (e.g., 30%, 40%, 50%, 60%, 70%, 80%, 90%, 95%, or the like) of the peak absorption. For instance, if the absorption spectrum indicates that the peak absorption of the material and/or the solution including the material is 0.4 at a wavelength of 750 nm, then the surface plasmon resonance band can be defined as a range of wavelengths (including 750 nm) corresponding to absorptions of 0.2 (i.e., 50% of the peak absorption) or greater.

In various cases, the surface plasmon resonance band of the nanoparticles 112 is defined according to an absorption spectrum (e.g., a UV-visible light-NIR (UV-VIS-NIR) absorption spectrum) of the nanoparticles 112 in a solution. The solution, for instance, may be the resin 102 itself, an aqueous solution of the nanoparticles 112 (e.g., a suspension of the nanoparticles 112 in water), or a construct that includes the nanoparticles 112.

When surface plasmon resonance of the nanoparticles 112 occurs, the energy from the excitation light is converted to heat. Accordingly, the nanoparticles 112 may be configured to convert light to heat. In various cases, the nanoparticles 112 may be referred to as “light-to-heat converting nanoparticles.” According to various implementations, the shape(s) of the nanoparticles 112 enable the nanoparticles 112 to convert the NIR light to heat, because the wavelength of the NIR light may correspond to (e.g., overlap) a plasmon resonance band of the nanoparticles 112.

In various implementations, at least one plasmon resonance band of the nanoparticles 112 corresponds to an NIR wavelength. The nanoparticles 112, for example, include nanorods, nanostars, nanospheres, nanocages, nanoclusters, nanoplates, nanotriangles, nanoshells, nanomatryoshkas, or any combination thereof.

The nanoparticles 112, in various cases, include one or more materials. Examples of materials in the nanoparticles 112 include gold, silver, titanium, nickel, silicon, carbon, graphene, one or more oxides thereof, one or more sulfides thereof, or any combination thereof. The nanoparticles 112, in some examples, are biocompatible. For instance, in particular cases, the nanoparticles 112 include biocompatible gold nanoparticles.

In various examples, a coating is disposed on surfaces of the nanoparticles 112. In some cases, the coating enhances the biocompatibility and/or stability of the nanoparticles 112. According to some examples, a PEG coating is disposed on the surfaces of the nanoparticles 112.

The nanoparticles 112 may be included in the resin 102 at a specific concentration. In general, the nanoparticles 112 can be included at a relatively low concentration in the resin 102. In various implementations, the nanoparticles 112 are 0.001% to 1% of the resin 102, by weight.

In various cases, the resin 102 may have various characteristics. For instance, the resin 102 may have a viscosity in a range of 0.25 Pascal second (Pa-s) to 10 Pa-s. In addition, the resin 102 may have optical characteristics that enable the resin 102 to be transparent to a frequency of light that activates the photoinitiator 110 and/or a frequency of light that is absorbed by the nanoparticles 112. For instance, the resin 102 may transmit and/or photocure at an electromagnetic wavelength of 250-800 nm. In various cases, the resin 102 is also transparent to other electromagnetic wavelengths, such as wavelengths in the NIR spectrum.

A hydrogel 114 may be generated by exposing the resin 102 to first light 116. A frequency of the first light 116 may depend on the photoinitiator 110 included in the resin 102. In various implementations, the first light 116 may have a wavelength in an ultraviolet (UV) spectrum. For instance, the wavelength may be in a range of 100 nm to 400 nm. In particular examples, the first light 116 has a wavelength in the UV-visible spectrum. For example, the wavelength of the first light 116 is in a range of 100 nm to 700 nm.

The hydrogel 114 may be generated by loading the resin 102 in a stereolithographic 3D printer 118 (also referred to as a “stereolithographic apparatus” or “SLA”). The 3D printer 118, for instance, includes a tank 120 configured to hold the resin 102. A platform 122 is at least partially disposed in the tank 120 and is configured to support the hydrogel 114 as it is generated in the tank 120.

The 3D printer 118 further includes a first light source 124 configured to emit the first light 116, as well as an optical system 126 configured to direct, focus, and reflect the first light 116 into the tank 120. In various implementations, the first light source 124 includes at least one laser. The optical system 126, for instance, includes one or more mirrors and/or one or more lenses. According to some examples, the optical system 126 further includes one or more actuators configured to reposition, turn, and otherwise move the mirrors and/or lenses. In various cases, a control system 128 is communicatively coupled to the first light source 124 and the optical system 126. The control system 128 can be implemented by at least one processor and memory storing instructions that, when executed by the at least one processor, cause the at least one processor to perform various operations. In some cases, the control system 128 is implemented by a computing system. According to various examples, the control system 128 controls the operation of the first light source 124 and/or optical system 126 in order to cause the first light 116 to enter the tank 120 in a particular pattern, thereby generating the hydrogel 114 in a predetermined 3D structure.

In various implementations, the first light 116 activates the photoinitiator 110, thereby causing the globular protein 106 and co-monomer 108 to polymerize in the tank 120. In various implementations, the co-monomer 108 binds to exposed methacrylated lysines in the globular protein 106. Further, methacrylated lysines of a first instance the globular protein 106 bind to methacrylated lysines of a second instance of the globular protein 106. Accordingly, the resin 102 is polymerized using the first light 116.

In some examples, the hydrogel 114 is generated without the use of the 3D printer 118. For instance, the hydrogel 114 can be fabricated by filling a mold with the resin 102 and exposing the resin 102 to the first light 116. According to some examples, a bioplastic 130 is generated by dehydrating the hydrogel 114. In some implementations, the bioplastic 130 is generated by drying the hydrogel 114. As used herein, the term “construct” may refer to the hydrogel 114 and/or the bioplastic 130.

In various cases, the bioplastic 130 is generated with a first shape. Subsequently, the bioplastic 130 is mechanically converted into a second shape. For example, an actuator 132 may deform the bioplastic 130 by applying a force 134 to the bioplastic 130. In various cases, the actuator 132 includes a press, a robotic arm, or some other mechanical device configured to apply the force 134. In some cases, the actuator 132 is a mechanical actuator, a linear actuator, a gripper, a hydraulic actuator, an electric actuator, or any combination thereof. The force 134, for instance, compresses, bends, twists, or a combination thereof, the bioplastic 130 in order to convert the bioplastic 130 into the second shape.

In various implementations, the bioplastic 130 includes different portions with different mechanical properties. These mechanical properties can be the result of different concentrations of elements within the different portions. For example, the bioplastic 130 may include a first portion with a first compressibility (e.g., due to a first ratio of globular protein to co-monomer) and a second portion with a second compressibility (e.g., due to a second ratio of globular protein to co-monomer). In various cases, the same force may cause the first portion of the bioplastic 130 to compress at a first percentage and may cause the second portion of the bioplastic 130 to compress at a second percentage. Accordingly, the bioplastic 130 may be reshaped in an irregular fashion due to the different mechanical properties of the different portions.

In particular cases, the second shape of the bioplastic 130 is smaller than the first shape of the bioplastic 130 in at least one dimension (e.g., a width or length). Accordingly, the bioplastic 130 may be capable of being inserted into a narrower opening in the second shape than if the bioplastic 130 is in the first shape. For instance, the bioplastic 130 may be inserted through a lumen or surgical port of a subject in the second shape, but the bioplastic 130 may be too large to be inserted through the lumen or surgical port in the first shape.

According to various implementations of the present disclosure, the first shape of the bioplastic 130 may be restored by exposing the bioplastic 130 to second light 136. In particular cases, the second light 136 is absorbed by the nanoparticles 112 in the bioplastic 130. A plasmon resonance band of the nanoparticles 112 (e.g., as measured in the resin 102 and/or bioplastic 130), for instance, is centered or otherwise overlaps a wavelength of the second light 136. The nanoparticles 112 in the bioplastic 130, for instance, release heat in response to absorbing the second light 136. The heat generated by the nanoparticles 112, in various implementations, relaxes the polymer in the bioplastic 130, thereby causing the bioplastic 130 to revert from the second shape to the original, first shape.

Although FIG. 1 illustrates a single type of second light 136, implementations are not so limited. In some examples, different portions of the bioplastic 130 may include different concentrations and/or types of the nanoparticles 112. For example, a first portion of the bioplastic 130 may include a first portion of the nanoparticles 112 having a first shape and/or length, and a second portion of the bioplastic 130 may include a second portion of the nanoparticles 112 having a second shape and/or length. Due to the different types of the nanoparticles 112, the shape of the first portion of the bioplastic 130 may be restored by a first wavelength of light (e.g., due to local heating caused by the first portion of the nanoparticles 112), whereas the shape of the second portion of the bioplastic 130 may be restored by a second wavelength of light (e.g., due to local heating caused by the second portion of the nanoparticles 112). Both of the first and second wavelengths may be NIR wavelengths, for instance. In some cases, multiple light sources may emit different types of light including the second light 136 in order to restore the bioplastic 130 to the first shape. In some examples, the bioplastic 130 is exposed to the different types of light at different times. Accordingly, the bioplastic 130 may be configured to unfold, or to otherwise restore the first shape, in a modular (e.g., multi-step) fashion.

In some cases, the second light 136 is transmitted through a biological tissue 138. For instance, the bioplastic 130 may be inserted into the body of a subject (e.g., a human) while the bioplastic 130 is in the second shape, and may be subsequently illuminated by the second light 136. Accordingly, the shape of the bioplastic 130 may be restored while the bioplastic 130 is disposed inside of the body of the subject. The second light 136, in various cases, has a wavelength that is transmissible to biological tissue. For example, the second light 136 may have a NIR wavelength.

The second light 136 may be delivered by a second light source 140. In various cases, the second light source 140 includes a laser, light-emitting diode (LED), or other type of light source. In examples in which the bioplastic 130 is inserted into the body of the subject, the second light source 140 may be disposed outside of the body of the subject. In some examples in which the bioplastic 130 is disposed in a lumen or cavity of the subject, the light source 140 may be disposed outside of the lumen or cavity.

The bioplastic 130 can be utilized for a variety of applications. For instance, the bioplastic 130 can be utilized in a synthetic graft, an implantable device, as a coating of an implantable device, in a surgical mesh, in a stent, in a patch, a bandage, or a microneedle structure. As used herein, the term “synthetic graft,” and its equivalents, may refer to a man-made material used to replace or support a biological tissue. Examples of synthetic grafts include synthetic bone grafts (e.g., including calcium phosphate-based structures that can serve as scaffolds for which cells attach and generate new bone tissue), artificial skin (e.g., including a collagen scaffold that induces skin growth), synthetic vascular grafts, synthetic intestinal mucosal grafts, and so on. In various implementations, the bioplastic 130 can be sutured to soft tissue, such as to skin, the abdominal wall, to blood vessel walls, a gastrointestinal (GI) tract, or the like. According to some cases, the bioplastic 130 degrades over time, such as when implanted in a subject.

In some implementations, the bioplastic 130 can include additional materials. According to some examples, a construct includes the bioplastic 130 as well as one or more additional materials, such as titanium, polyvinylchloride (PVC), polypropylene, polyethylene terephthalate (PET), polytetrafluorethylene (PTFE), polymethylmethacrylate (PMMA), stainless steel, silicone, or a ceramic. In some examples, the bioplastic 130 includes a therapeutic agent. Examples of the therapeutic agent include therapeutic proteins (e.g., antibody-based biologics, Fc fusion proteins, blood factors, growth factors, hormones, interleukins, etc.), antibiotics (e.g., cephalosporins, glycopeptides, lincomycins, macrolides, quinolones, sulfonamides, tetracyclines, etc.), and anti-inflammatory agents (e.g., corticosteroids, such as cortisone, prednisone, and methyl prednisolone; non-steroidal anti-inflammatory drugs (NSAIDs), such as aspirin and ibuprofen; anti-tumor necrosis factor alpha (anti-TNF) biologics, such as adalimumab, certolizumab pegol, etanercept, golimumab, and infliximab)). When the bioplastic 130 degrades over time (e.g., while being disposed inside the body of a subject), the therapeutic agent is released. In some examples, a construct including the bioplastic 130 includes engineered microbes that release a therapeutic agent. Accordingly, in various implementations, the bioplastic 130 can be utilized to deliver a therapeutic agent to a subject, such as when the construct is implanted into the subject.

In various implementations, a surface of the bioplastic 130 is functionalized. For instance, the surface could be chemically modified and/or a material can be adsorbed to the surface. In some examples, the surface of the bioplastic 130 is treated to make it hydrophobic. For instance, silylation (e.g., using a hydrophobic silylating agent) may be performed on the surface of the bioplastic 130 or a plasma treatment may be performed on the surface of the bioplastic 130 (e.g., in order to oxidize the surface). In various implementations, the surface is modified to fine-tune desired mechanical properties of the bioplastic 130, to allow cells to adhere to the surface, to prevent cells from adhering to the surface, or to prevent diffusion of water or nutrients to other structures. In some cases, the surface is modified to enhance the biocompatibility of the bioplastic 130.

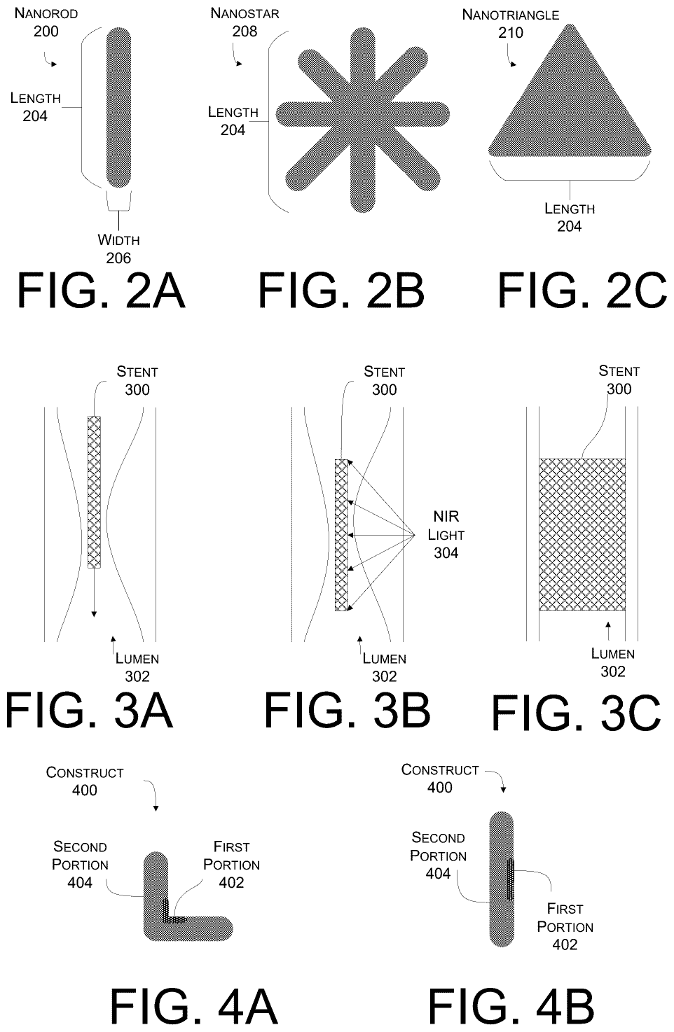

FIGS. 2A to 2C illustrate examples of various types of nanoparticles that can be used in various types of shape-restoring materials described herein. For instance, the nanoparticles illustrated in FIGS. 2A to 2C may include gold and/or silver. FIG. 2A illustrates an example of a nanorod 200 having a width 202 and a length 204. In various examples, the nanorod 200 has a tubular structure, such that the width 202 is a diameter of the nanorod 200.

In various implementations of the present disclosure, the width 202 and/or the length 204 correspond to a plasmon resonance band matching (e.g., overlapping) the wavelength of light that is absorbed by the nanorod 200. For example, the nanorod 200 may absorb light at the wavelength and convert energy from the light into heat. In various cases, the width 202 and/or the length 204 correspond to a plasmon resonance band with an NIR wavelength.

FIG. 2B illustrates an example of a nanostar 206 having the length 204. In various implementations, the nanostar 206 may have greater than 3 arms extending from a portion (e.g., a center portion) of the nanostar 206. FIG. 2B, for example, illustrates a cross-section of the nanostar 206, wherein the cross-section is through eight arms of the nanostar 206. In various cases, the length 204 extends along two arms of the nanostar 206. In certain implementations, the length 204 may extend along a single arm of the nanostar 206. Although not specifically illustrated in FIG. 2B, in some cases, the nanostar 206 includes arms of different lengths including the length 204. In various implementations of the present disclosure, the length 204 corresponds to plasmon resonance of the nanostar 206 that occurs with excitation light having an NIR wavelength, and which is absorbed by the nanostar 208. For example, the nanostar 208 may absorb light at the wavelength and convert energy from the light into heat.

FIG. 2C illustrates an example of a nanotriangle 208 having the length 204. For example, the nanotriangle 208 includes three sides that each have the length 204. According to various cases, the nanotriangle 208 has a plate shape. Although FIG. 2C illustrates a particular plate shape, implementations of the present disclosure are not so limited. Other potential plate shapes include squares, pentagons, hexagons, and other polygonal shapes.

FIGS. 3A to 3C illustrate an example of the use of a stent 300 that includes a shape-restoring material. For example, the stent 300 may include a construct and/or bioplastic described herein. In some cases, the stent includes a lattice structure including the construct and/or bioplastic. The stent 300 is biocompatible. For example, the stent 300 may include a construct that includes gold nanoparticles.

FIG. 3A illustrates the stent 300 being inserted into a lumen 302. For example, the stent 300 may be in a compressed form, such that a diameter of the stent 300 is smaller than a diameter of the lumen 302. In various cases, the lumen 302 is at least partially constricted. The lumen 302, for instance, is a blood vessel (e.g., an artery), a ureter, or a gastrointestinal tract (e.g., an esophagus). For instance, the lumen 302 is constricted due to the presence of a tumor, inflammation, plaque, or scar tissue.

FIG. 3B illustrates the stent 300 being illuminated with NIR light 304 while the stent 300 is disposed in the lumen 302. As shown, the NIR light 304 is transmitted through at least a portion of a tissue bordering the lumen 302. In various cases, nanoparticles in the stent 300 are configured to absorb the NIR light 304 and convert energy from the NIR light 304 into heat. The heat is released locally into the stent 300, causing the stent 300 to revert to an expanded shape. In some cases, the NIR light 304 is pulsed at a duty cycle that prevents cellular damage of the tissue bordering the lumen 302.

FIG. 3C illustrates the stent 300 in the lumen 302 after being illuminated with the NIR light 304. The stent 300 is in the expanded shape, which broadens an internal diameter of the lumen 302. For example, the stent 300 may have a hollow tubular shape, such that fluids may freely move through the lumen 302 in the expended shape. In various cases, the stent 300 is configured to dissolve while being disposed in the lumen 302. In some implementations, the stent 300 (e.g., the shape-restoring construct in the stent 300) includes a therapeutic, such as an anti-tumor or anti-inflammatory agent. Accordingly, if at least a portion of the stent 300 dissolves or otherwise degrades after being inserted into the lumen 302, the therapeutic may be delivered to a subject that includes the lumen 302.

FIGS. 4A and 4B illustrate an example of a construct 400 that can be selectively bent and straightened using a shape-restoring material. The construct 400 includes a first portion 402 and a second portion 404. In various cases, the first portion 402 and the second portion 404 include different concentrations of constituent elements. For example, the first portion 402 may include a different concentration of a globular protein, co-monomer, nanoparticles, or any combination thereof, to the second portion 404. In some cases, the first portion 402 is more compressible than the second portion 402. For example, when a force is applied to the construct 400, the construct bends at the first portion 402. In some implementations, the first portion 402 includes a higher fraction of globular protein than the second portion 404. In some examples, the first portion 402 includes a lower fraction of co-monomer than the second portion 404.

In various cases, the first portion 402 includes a different type of nanoparticles than the second portion 404. For example, the nanoparticles in the first portion 402 absorb light at a first wavelength, and the nanoparticles in the second portion 404 absorb light at a second wavelength. Thus, in some cases, the construct 400 can be unfolded by irradiating the construct 400 with the light having the first wavelength, such that the first portion 402 expands to a previous shape without the second portion 404 expanding to a previous shape.

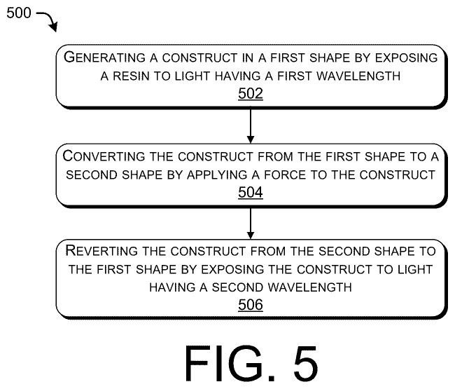

FIG. 5 illustrates an example process 500 for utilizing a shape-restoring material. The process 500, for instance, can be performed by an entity including a SLA, one or more light sources, one or more actuators, or any combination thereof. At 502, a construct is generated in a first shape by exposing a resin to light having a first wavelength. In various cases, the resin includes a globular protein, a co-monomer, nanoparticles, a photoinitiator, and water. The resin, for instance, has a viscosity in a range of 0.25 Pa*s to 10.0 Pa*s. In some implementations, the construct is generated by an SLA printer. The construct, for instance, is a bioplastic.

The globular protein, for instance, includes at least one of a serum albumin (e.g., BSA and/or MABSA), pepsin, hemoglobin, lysozyme, lactoglobulin, pea protein, or soy protein. In some examples, the resin includes 1% to 50% of the globular protein by weight. For instance, the resin includes 20% to 40% or 25% to 30% of the globular protein by weight.

The co-monomer, in various cases, includes at least one of PEGDA, HEA, or AAm. According to various examples, the co-monomer is water-soluble. In various cases, the resin includes 1% to 60% of the co-monomer by might. For instance, the resin includes 5% to 20% or 10% to 15% the co-monomer by weight.

According to various implementations, the nanoparticles are light-to-heat converting nanoparticles. In some cases, the nanoparticles include a metal (e.g., gold and/or silver) and/or carbon (e.g., graphene). The nanoparticles, for instance, have a coating (e.g., including PEG). The nanoparticles may include at least one of nanorods, nanospheres, nanocages, nanoclusters, nanoplates, nanotriangles, or nanoshells. According to various cases, a plasmon resonance band of the nanoparticles may correspond to (e.g., overlap) at least one NIR wavelength. For instance, the nanoparticles may be configured to generate heat in response to being exposed to NIR light. In some examples, the nanoparticles have a length of 60 nm and/or a width of 15 nm. In various cases, the resin includes 0.001% to 1% the nanoparticles by weight.

The photoinitiator is configured to polymerize the globular protein and the co-monomer in response to being exposed to light. In some examples, the photoinitiator includes at least one of LAP, Ru(bpy)3 and SPS, or 2-hydroxy-2-methylpropiophenone. The light that causes the photoinitator to polymerize the globular protein and the co-monomer, for instance, is UV-visible light.

In some implementations, at least a portion of the water is removed from the construct. For example, the construct is dried.

In various implementations, different portions of the construct have different concentrations of constituent materials and/or different physical properties. For example, different portions of the construct may have different amounts and/or ratios of the globular protein, the co-monomer, the nanoparticles, the photoinitiator, or any combination thereof. In various cases, different portions of the construct have different elasticities, compressabilities, transmittance, or other physical properties.

At 504, the construct is converted from the first shape to a second shape by applying a force to the construct. In some cases, the construct is compressed, bent, twisted, or otherwise shaped. The second shape may have at least one shorter dimension than the first shape. For example, a width of the second shape may be 60% to 70% of a width of the first shape. In various cases, the construct may be moved through a relatively narrow space while the construct is in the second shape. For example, the construct is optionally moved into a lumen or space within a body of a subject while the construct is in the second shape.

At 506, the construct is reverted from the second shape to the first shape by exposing the construct to light having a second wavelength. According to various implementations, the light having the second wavelength is absorbed by the nanoparticles in the construct. The absorbed energy is released by the nanoparticles as heat. As a result of the heat, the construct is transformed from the second shape back to the first shape. In some examples, the construct is exposed to the light in pulses. The frequency and/or duty cycle of the pulses may be controlled to prevent excessive heating of the construct. For example, a temperature sensor may be used to detect a temperature of the construct. In various cases, a duty cycle of the pulses may be reduced, or the light may be at least temporarily turned off, when the temperature of the construct exceeds a threshold temperature.

In various implementations, the second wavelength is an NIR wavelength. For instance, the second wavelength is in a range of 700 nm to 800 nm. According to some cases, the light is transmitted through at least one biological tissue. For example, if the construct is disposed inside a body of a subject, a source of the light may be disposed outside of the body of the subject and the light is transmitted through at least a portion of the body of the subject. In particular examples, the construct is included in a stent, such as a stent for placement in a gastrointestinal tract of the subject.

### EXPERIMENTAL EXAMPLE

4D printing is the 3D printing of objects that can change chemically or physically in response to an external stimulus. These objects are attractive for a wide range of applications that include robotics, aeronautics, and medicine. Photothermally responsive shape memory materials are highly attractive for their ability to undergo remotely activated shape recovery. While photothermal methods using gold nanorods (AuNRs) constitute a highly attractive form of indirect heating for shape recovery, 3D patterning of these materials into more complex object geometries is a significant challenge. The present disclosure describes techniques to fabricate 3D printed shape memory bioplastics with photo-activated shape recovery.

In this experimental example, protein-based nanocomposites based on bovine serum albumin (BSA), poly (ethylene glycol) diacrylate (PEG-DA) and gold nanorods (AuNRs) were developed for stereolithographic apparatus (SLA) 3D printing. These 3D printed bioplastics can be mechanically deformed under high loads, and the proteins serve as mechanoactive elements that unfold in an energy-dissipating mechanism that prevents fracture of the thermoset. The bioplastic object remained in this metastable state under ambient conditions. 98% photothermal shape recovery under near-infrared light was achieved within two minutes due to the significant amount of heat produced with a low concentration (e.g., 0.0025% by weight) of AuNRs, and the recovery was achieved without changes to the mechanical performance of the shape memory material in a subsequent cycle. Selective recovery can also be achieved with spatially controlled irradiation of bioplastic constructs. These shape memory thermoset composites are suitable for the fabrication of biodegradable shape-morphing biomedical devices.

The present disclosure describes techniques to fabricate 3D printed protein-based nanocomposites with remote photothermal shape recovery. To achieve the indirect light activation, a low amount of gold nanorods (0.001% wt) that are tuned with the main LSPR at 795 nm (longitudinal mode) and transversal mode at 509 nm were incorporated into a BSA based protein/bolymer matrix. Mechanically deformed 3D-printed bioplastics can return to their original shape under the irradiation of the near-infrared (NIR) laser (808 nm) at 3.75 W cm⁻². Under the irradiation of NIR, gold nanorods in the bioplastic convert enough heat from light to raise the temperature above the glass transition temperature (Tg) of the bioplastic to facilitate the recovery process. In this experimental example, selective recovery was achieved with controlled localized irradiation and shape transformation could be triggered remotely under pork gelatin skin without damage to the skin. The disclosed BSA-based bioplastic can be used as material for deployable in vivo biomedical devices (e.g., stents).

### Experimental Procedures

Materials: Poly (ethylene glycol) (PEG) diacrylate (Mn 700 DA), gelatin from porcine skin were purchased from Sigma-Aldrich Corp. (St. Louis, MO). Ultrapure and ultralow fatty acid content BSA was purchased from NOVA Bidlogics, Inc. (Oceanside, CA). All reagents were used as received. Methacrylate bovine serum albumin (MABSA) was synthesized according to a reported procedure (Smith et al., Biomacromalecules 2020, 21, 484). AuNRs PEG was synthesized following a protocol published by González-Rubio et al., ACS Nano 2019, 13, 4424, using hexadecyltrimethylammonium bromide (CTAB 96%), 1-decand (n-decanol, 98%), hydrogen tetrachloroaurate trihydrate (HAuCl₄·3H₂O, ≥99.9%), silver nitrate (AgNO₃, ≥99.0%), L-ascorbic acid (≥99%), and sodium borohydride (NaBH₄, 99%), thiol-terminated PEG (O-(2-(3-mercaptopropionylamino)ethyl]-O′-methylpolyethylene glycol (MW 5000 g/mol), which were obtained from Merck & Co., Inc. of Rahway, NJ. MilliQ grade water (resistivity 18.2 MΩ cm at 25° C.) was used.

Synthesis of AuNRs PEG: AuNRs (60 nm length, 15 nm width), with the main LSPR at 795 nm (longitudinal mode) and another one at 509 nm (transversal mode), were synthesized by following a protocol published by Gonzãlez-Rubio et al. The synthesized AuNRs were centrifuged at 8965 g for 20 min twice to remove the excess CTAB as well as other reagents and redispersed in 1 mM CTAB at a final concentration of 10.95 mM (Au(0)). Then 500 μL of AuNR solution was again centrifuged at 8965 g for 10 min, decanted, and 1 mL of 0.1 mM thiol-terminated PEG (O-(2-(3-mercaptopropionylamino)ethyl]-O′-methylpolyethylene glycol (MW 5000 g/mol) was added dropwise under sonication. The mixed solution was shaken for 2 hours at room temperature and centrifuged twice at 8965 g for 10 min, decanted, and resuspended in water to remove excess of PEG reagent.

Preparation of BSA-based or MABSA-based resin for vat photopolymerization: The weight percentages described herein are based on the total composition of the resin, including the aqueous solvent. In this experimental example, the preparation of the 5 g of resin with 30 wt % of BSA, 10 wt % poly(ethylene) diacrylate (PEG-DA) and 0.0015% AuNRs is described. First, 126.7 μL of AuNRs was added in to 2873.3 μL of DI water, then 0.5 g of PEG-DA was dissolved in the solution. 1.5 g of BSA or MABSA was slowly added to the solution with mixing until dissolved. Next, 50 mg of LAP was added to the resin with mixing until dissolved. The final resin formulation was covered with foil and stored at least 12 hours for SLA 3D printing. The comonomer, AuNRs, and DI water quantities were changed to prepare other formulations.

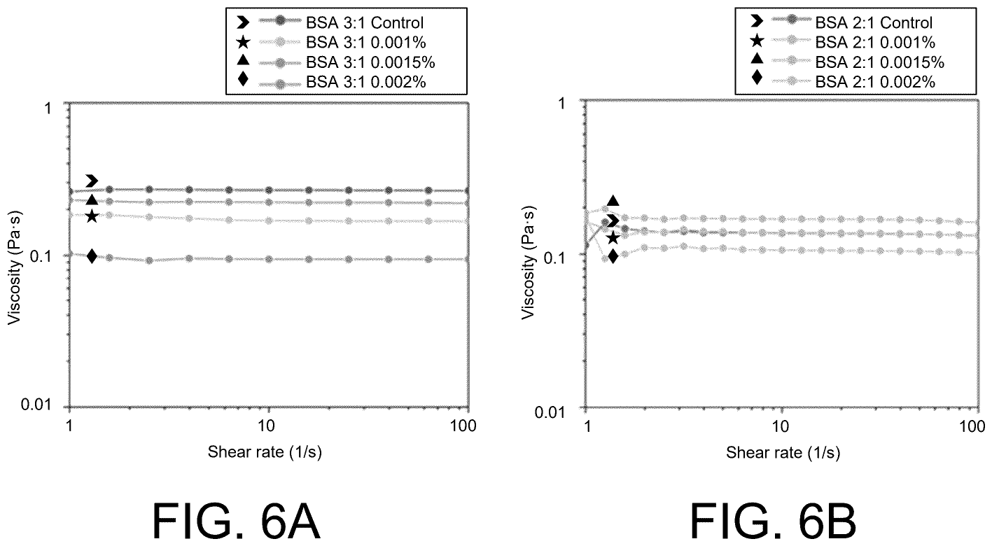

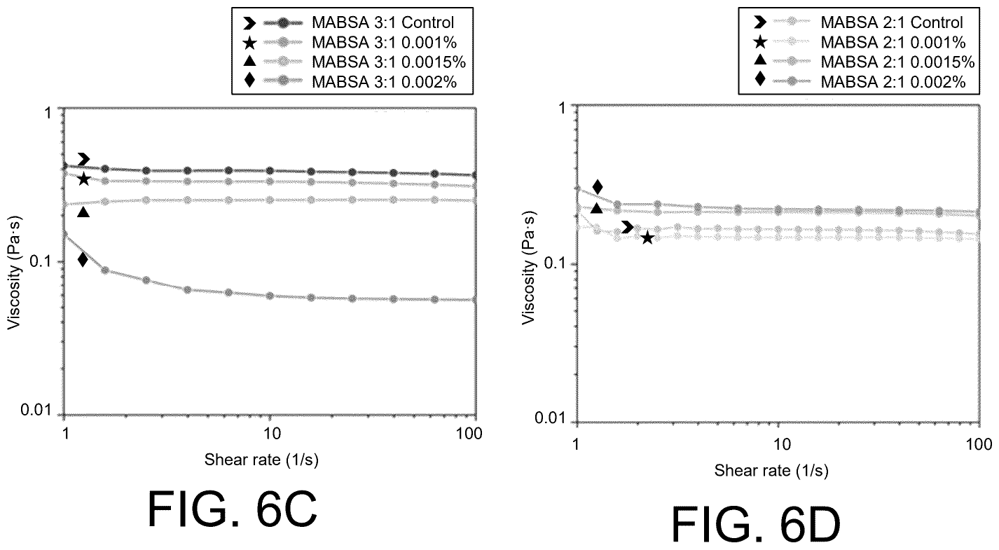

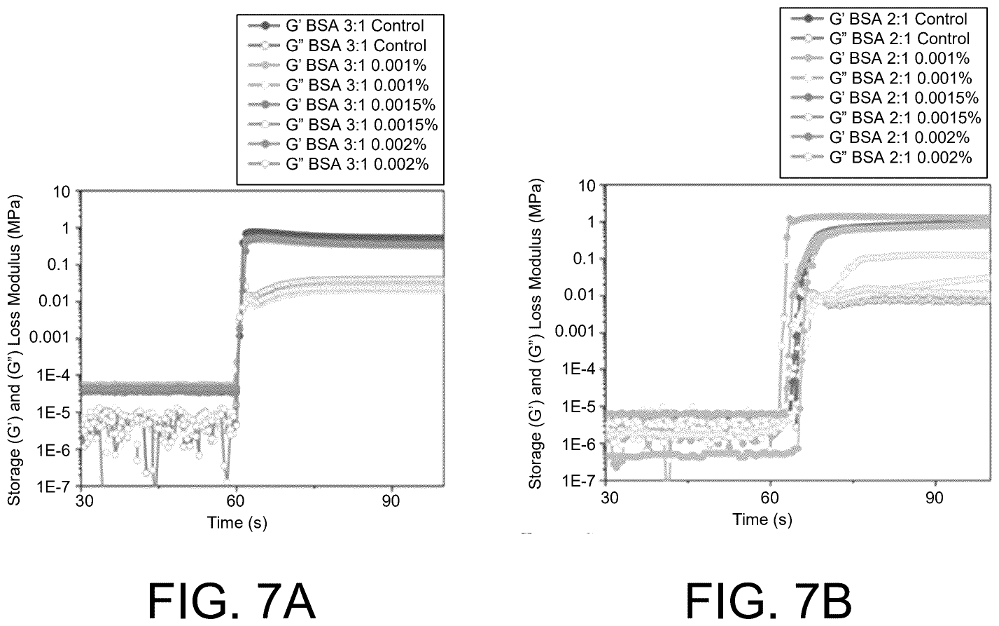

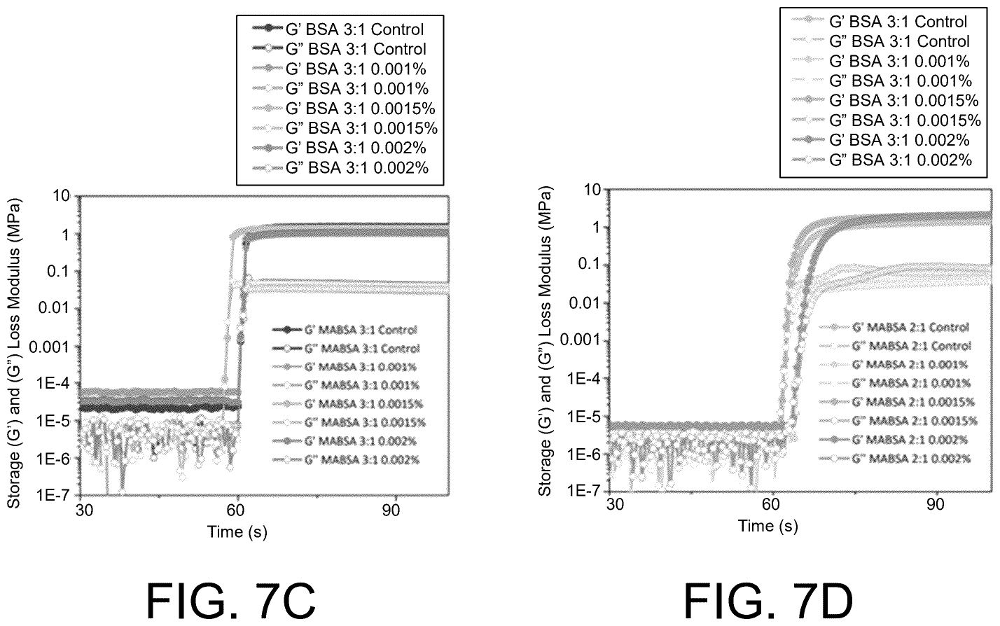

Rheology Measurements: A Discovery Hybrid Rheometer-2 (from TA Instruments of New Castle, DE) was used to conduct viscosity test and photo rheology experiments. For viscosity vs shear rate experiment (FIGS. 6A to 6D), shear rate was increased from 1 to 100 s⁻¹ using a 40 mm cone and plate geometry 1.019, a solvent gap, and a gap height of 26 μm. For photo rheology experiments (FIGS. 7A to 7D), a 365 nm LED UV-curing accessory with disposable acrylic plate was used. The tests were conducted using constant 1% strain and a frequency of 1 Hz with a gap height of 1000 μm. A 60 second dwell time elapsed before the UV light was turned on for 120 seconds.

FIGS. 7A to 7D illustrate rates of photocuring of examples resin determined using a rheometer. FIG. 7A illustrates photorehometry for BSA 3:1 resin with 0-0.002% of AuNRs; FIG. 7B illustrates photorehometry for BSA 2:1 resin with 0-0.002% of AuNRs; FIG. 7C illustrates photorehometry for MABSA 3:1 resin with 0-0.002% of AuNRs; and FIG. 7D illustrates photorehometry for MABSA 2:1 resin with 0-0.002% of AuNRs.

3D printing: A Form 2 printer (from Formlabs of Somerville, MA) with 405 nm violet laser (250 mW) was used to fabricate the 3D hydrogel constructs. To reduce the total volume of resin for printing, the build plate and resin tray were modified. The 3D hydrogel constructs were designed with Fusion 360 (from Autodesk of San Francisco, CA). Resin was slowly poured into the reservoir, and the 3D hydrogel constructs were then printed using the open mode on the Form 2 printer with a layer height of 100 μm. After the printing process was completed, the 3D hydrogel constructs were removed from build plate using a razor blade, and then rinsed in deionized (DI) water to remove any uncured resin. The 3D printed hydrogel constructs were air dried for 48 hours to transfer to bioplastics. The naming and components of different formulated bioplastics are summarized in Table 1.

Thermal Analysis: Glass transition temperature was tested using a Toledo DSC 3+ differential scanning calorimeter (from Mettler-Toledo of Columbus, OH). Each sample weights between 5-10 mg. All scans were carried out in hermetic aluminum pan under nitrogen atmosphere from −80 to 200° C. with a scan rate of 10° C./min. The glass transition temperatures for different formulated bioplastic are given in Table 1.

TABLE 1
Formulations, naming, and glass transition
temperature (Tg) for different bioplastics.

| | | PEG-DA | Gold | |
|---|---|---|---|---|
| | | (700 g/mol) | Nanorod | T_{g} |
| Name | Protein % | % | (AuNR) % | (° C.) |
| BSA 2:1 Control | BSA 66.7 | 33.3 | 0 | 46.1 |
| BSA 2:1 0.0025 | BSA 66.7 | 33.3 | 0.0025 | 48.4 |
| BSA 2:1 0.00375 | BSA 66.7 | 33.3 | 0.00375 | 46.2 |
| BSA 2:1 0.005 | BSA 66.7 | 33.3 | 0.005 | 44.2 |
| BSA 3:1 Control | BSA 75 | 25 | 0 | 51.2 |
| BSA 3:1 0.0025 | BSA 75 | 25 | 0.0025 | 51.5 |
| BSA 3:1 0.00375 | BSA 75 | 25 | 0.00375 | 50.1 |
| BSA 3:1 0.005 | BSA 75 | 25 | 0.005 | 49.5 |
| MABSA 2:1 Control | MABSA 66.7 | 33.3 | 0 | 34.3 |
| MABSA 2:1 0.0025 | MABSA 66.7 | 33.3 | 0.0025 | 35.5 |
| MABSA 2:1 0.00375 | MABSA 66.7 | 33.3 | 0.00375 | 36.8 |
| MABSA 2:1 0.005 | MABSA 66.7 | 33.3 | 0.005 | 37.6 |
| MABSA 3:1 Control | MABSA 75 | 25 | 0 | 40.5 |
| MABSA 3:1 0.0025 | MABSA 75 | 25 | 0.0025 | 42.7 |
| MABSA 3:1 0.00375 | MABSA 75 | 25 | 0.00375 | 42.6 |
| MABSA 3:1 0.005 | MABSA 75 | 25 | 0.005 | 41.3 |

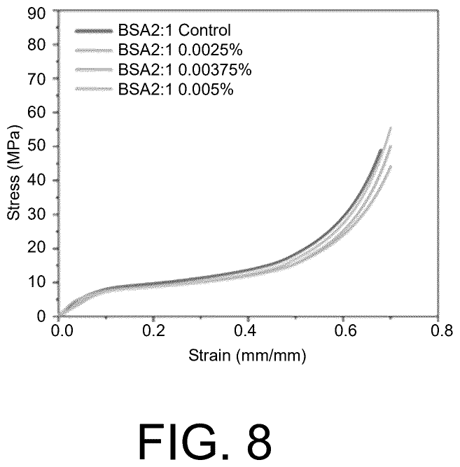

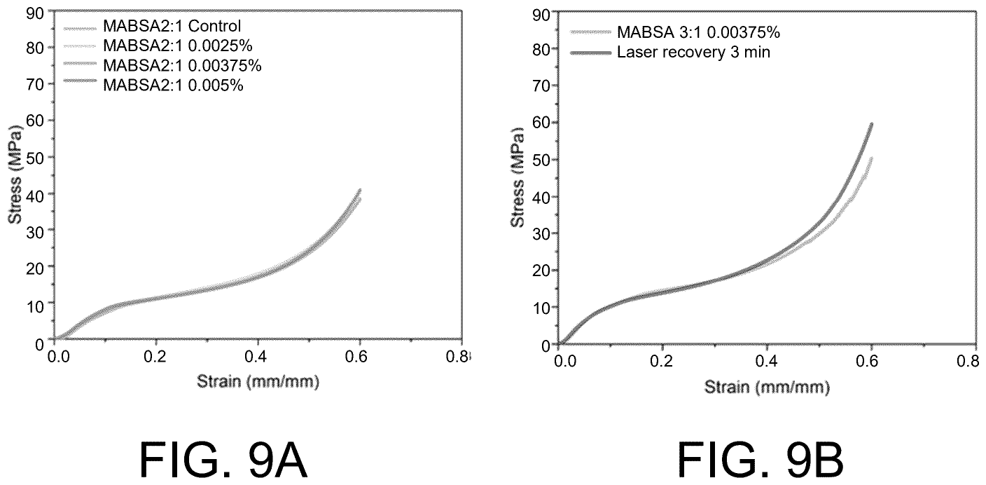

Mechanical Properties: Uniaxial compression tests were performed using Instron 5585H 250 kN electromechanical test frame with a 50 kN load cell. Cylindrical bioplastic compression samples (5.2 mm diameter×8.3 mm height) were compressed using a crosshead rate of 1.3 mm/min until 70% strain for BSA-based bioplastic (FIG. 8) and 60% stain for MABSA-based bioplastic (FIGS. 9A and 9B). The moduli were determined from the resulting stress-strain curve, and compressive moduli were calculated from the slope of elastic region of the stress-strain curve by OriginLab.

FIG. 8 illustrates example compressive stress vs strain curves of 3D printed BSA 2:1 bioplastics with different concentrations of AuNRs.

FIGS. 9A and 9B illustrate example compressive stress vs strain curves of 3D printed bioplastics. FIG. 9A illustrates results of an example MABSA 3:1 bioplastic with different concentrations of AuNRs. FIG. 9B illustrates results of an example MABSA 3:1 0.00375% bioplastic compared with its laser-recovered bioplastic.

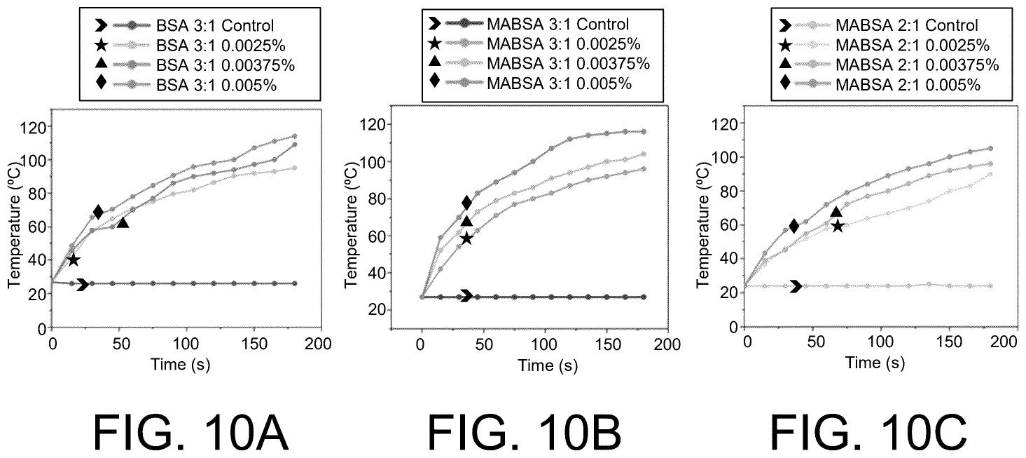

Photothermal Performance and NIR Light Actuation: Infrared diode laser (MDL-Ill-808-2.5 W) (from Opto Engine LLC of Midvale, UT) was used as 808 nm laser source for actuation. The power intensity of the laser was ≈3.75 W cm⁻² for both global and localized actuation. The dimensions of the beam at aperture are ˜5 mm×8 mm. FLIR TG165-X thermal camera was used to measure the temperature vs irradiation time, the temperature was recorded for 3 min for different bioplastics. FIGS. 10A to 10C illustrate thermal curves of examples of the 3D printed bioplastic recovery process under 3-minute irradiation of NIR laser (808 nm). FIG. 10A shows a time vs temperature curve for an example of BSA 3:1 0-0.005% AuNRs. FIG. 10B shows a time vs temperature curve for an example of MABSA 3:1 0-0.005% AuNRs. FIG. 10C shows a time vs temperature curve for an example of MABSA 2:1 0-0.005% AuNRs.

Preparation of Pork Gelatin Skin: 5 g of gelatin powder from porcine skin was mixed with 50 mL of DI water in a 200 mL beaker. Then, the mixture was heated to 60° C. while stirring until the gelatin powder was well dissolved. Orange food coloring was added to make the solution nontransparent. The final warm solution was poured into a circular mold (60 mm diameter×3.25 mm height), then placed on a bench at room temperature for 4 hours.

### Results and Discussion

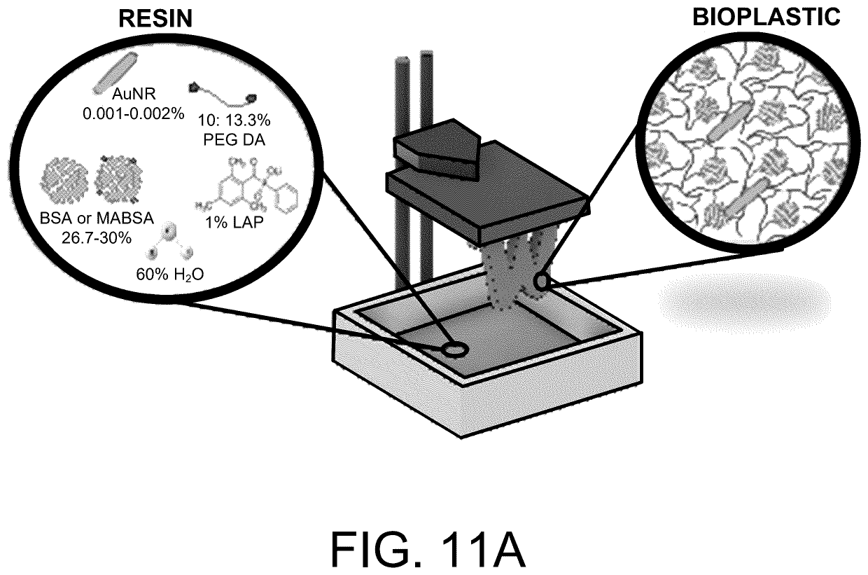

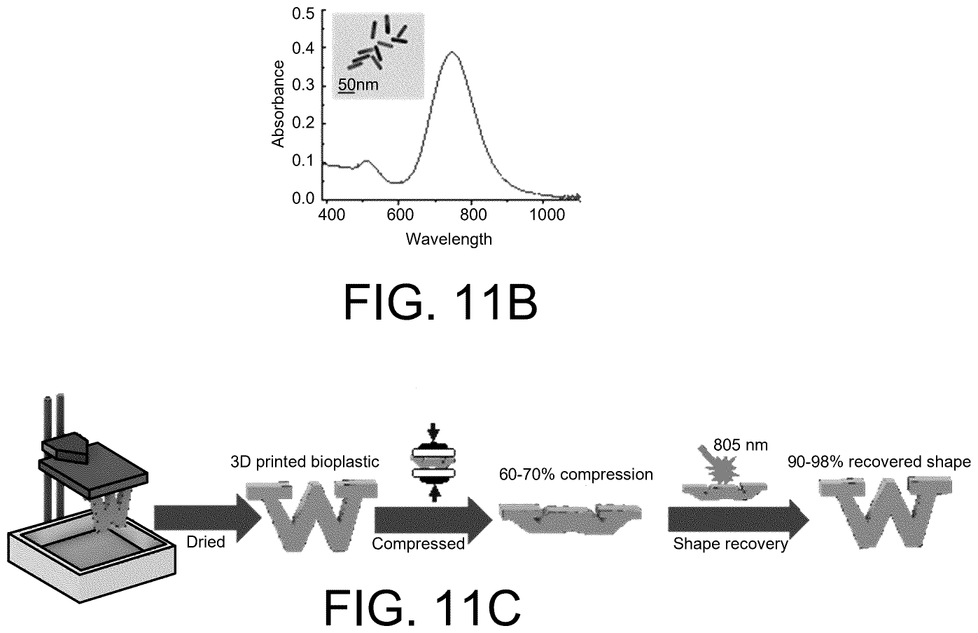

FIG. 11A is a schematic illustration of the SLA 3D printing of the AuNRs incorporated BSA or MA-BSA based resin and the molecular level structures of the bioplastic. FIG. 11 B illustrates the UV-VIS-NIR spectrum of the synthesized gold nanorods used in this example, with an insert showing a TEM image of PEG-coated AuNRs in water (Scale bar, 50 nm). FIG. 11C illustrates a general scheme for the workflow utilized in this example. The “W” shape was 3D printed and then dried for 48 hours to transform to a bioplastic. The bioplastic was physically compressed to its secondary structure and then recovered its original shape upon irradiation to NIR light.

FIG. 11A illustrates stereolithography (SLA) 3D printing of the photothermal responsive AuNRs/BSA-based resin composition and its molecular level structure in the 3D printed bioplastics. Previous research reported BSA/PEG-DA resin formulations (E. Sanchez-Rexach, et al., ACS Appl. Mater. Interfaces 2021, 13, 19193). In this formulation, the surface lysine groups of BSA were reacted with the acylate group on PEG-DA via aza-Michael addition. A methacrylate bovine serum albumin (MA-BSA)/PEG-DA formulation has been previously reported (P. T. Smith, et al., Biomacromolecules 2020, 21, 484), where >90% of the surface lysine groups on BSA were converted into methacrylamide derivatives to form MABSA. The resins were printed using an SLA Form 2 printer, which utilized a 405 nm laser to photopattern the 3D object. The printing process of BSA/PEG-DA resin involved the polymerization of the free acrylate groups of PEG-DA to create a cross-linked network, while the MABSA/PEG-DA resin involved the polymerization of the free acrylates group of PEG-DA and methacrylate group of MA-BSA to create a network. In this work, 3:1 BSA, MABSA, and 2:1 BSA, MABSA resin formulation was chosen, which contained 26.7-30 wt. % protein, 10-13.3 wt. % PEG-DA (700 g/mol), 60 wt. % of DI water, 1 wt. % of LAP, and 0.001%-0.002 wt. % of gold nanorods. Table 1 provides the compositions and naming of bioplastics used in this study.

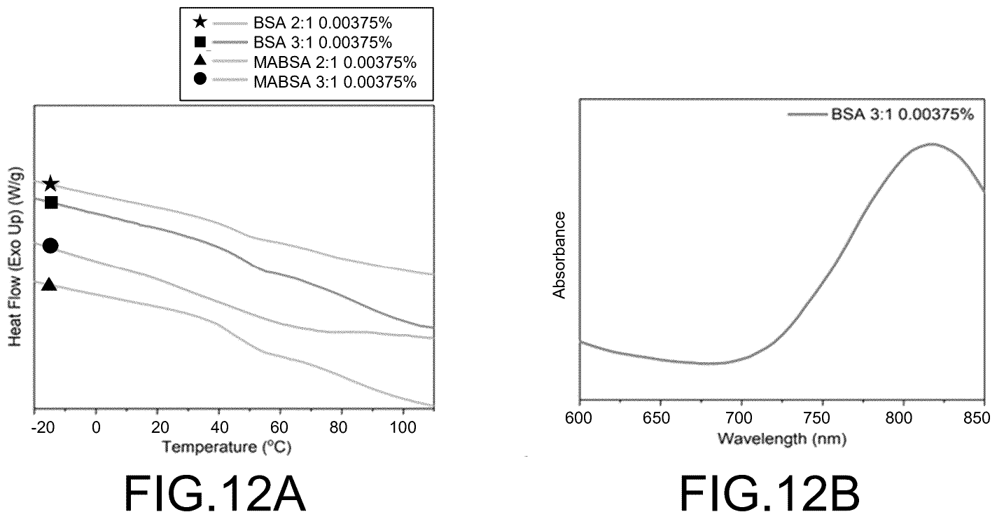

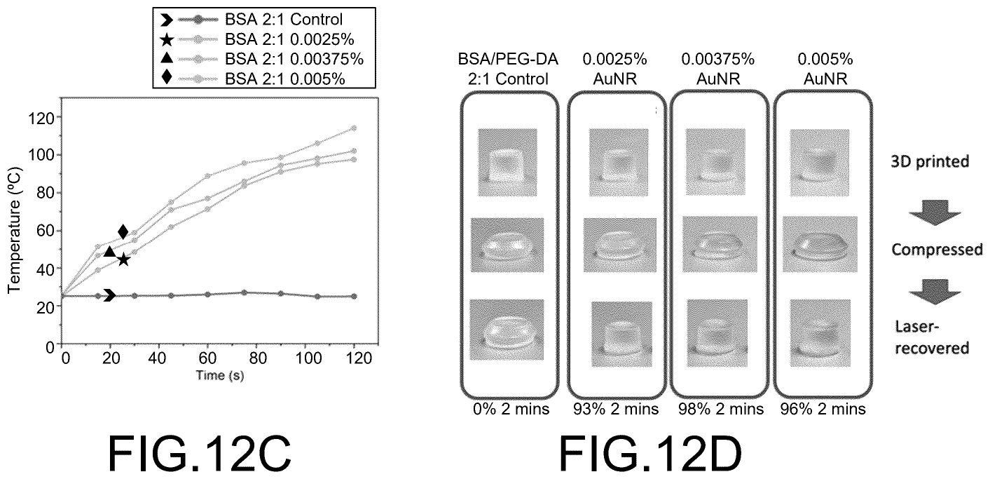

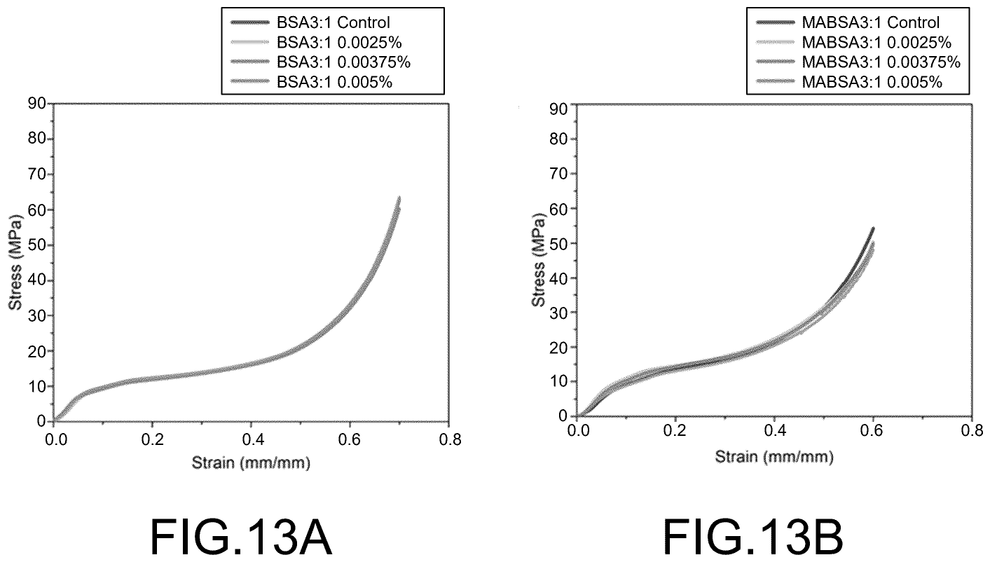

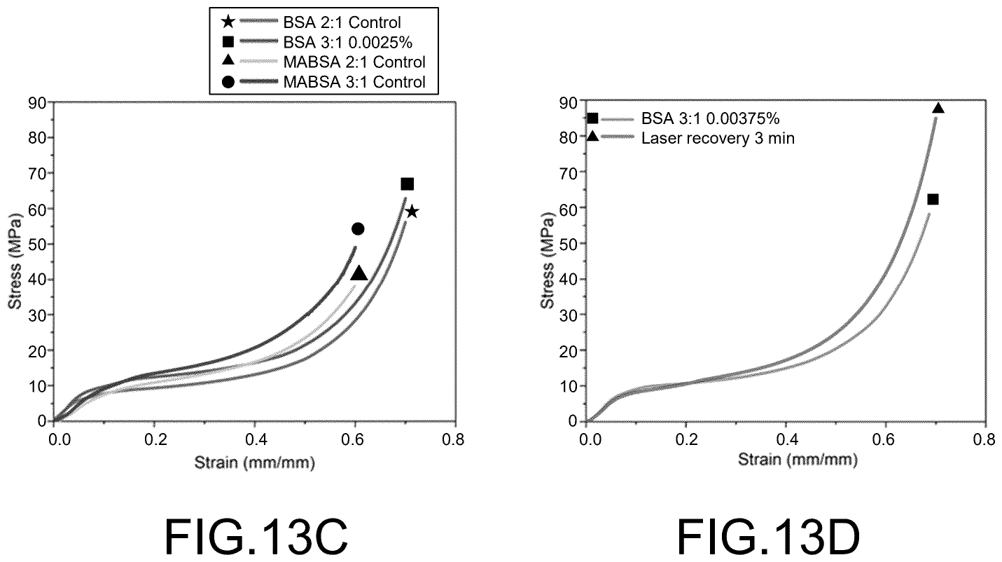

FIG. 11B depicts the UV-VIS-NIR spectrum of the synthesized gold nanorods, which exhibited two distinct peaks at 795 nm and 509 nm in this experimental example, corresponding to main LSPR at longitudinal mode and transversal mode of the gold nanorods, respectively. The TEM image of the synthesized gold nanorods demonstrated excellent monodispersity, no aggregation, and well-defined rod shape. To ensure the AuNR-incorporated resins have good printability in SLA printer, the viscosity and the photocuring rate of the resin were evaluated using the rheometer. Based on previous studies, the ideal viscosity ranges are from 0.25 to 10 Pa-s for resins to be able to reflow for each level of printing (E. Sanchez-Rexach, et al., ACS Appl. Mater. Interfaces 2021, 13, 19193). The viscosities of the 16 different formulations tested in this experimental example were well below 1 Pa-s, as shown in FIGS. 12A to 14B. Additionally, the photopolymerization kinetics of the resin were studied using real time photorheology, where the evolution of both storage and loss modulus was monitored during UV irradiation. The sudden increase in modulus observed upon turning on the UV light (at 60 seconds) demonstrates high light sensitivity of the resin, indicating its suitability for SLA 3D printing, as shown in FIGS. 13A to 15D.

The general scheme for the workflow is depicted in FIG. 11C. First, the 3D printed hydrogel was dried for a period of 48 hours under room temperature and atmospheric pressure to facilitate its transfer to bioplastic. Subsequently, the bioplastic underwent a physical compression process that reduced its original shape by 70% (for BSA bioplastic) and 60% (for MABSA bioplastic), using a load frame. The compressed structures were then subjected to irradiation using a near-infrared (NIR) laser with power of 3.75 W cm⁻². The irradiation was stopped when the compressed structure has fully recovered to its original shape, which took approximately 2-3 minutes.

FIG. 12A illustrates differential scanning calorimetry (DSC) curves of 0.0035% example AuNRs bioplastics. FIG. 12B illustrates the UV-VIS spectrum for an example 3D-printed BSA 3:1 0.00375% film. FIG. 12C illustrates a temperature vs irradiation time graph for four different example formulated bioplastics with 0.0035% AuNRs. FIG. 12D illustrates a diagram for original, compressed, and laser recovered shape (Disk) for 0-0.002% AuNRs in examples of a BSA 2:1 formulated bioplastic.

DSC was used to measure the glass transition temperature (Tg) values of four different bioplastics containing 0.00375% AuNRs, as shown in FIG. 12A. The Tg values of BSA 2:1 and 3:1 0.0035% bioplastic were 46.21° C. and 50.13° C., respectively. The Tg values of MABSA 2:1 and 3:1 0.0035% were 38.06° C. and 42.63° C., respectively. The Tg of a miscible mixture can be predicted by Flory-Fox equation, which is based on the weight fraction of the components (S. Páztor, et al., Materials 2020, 13, 4822). Since both BSA and MABSA have higher Tg than PEG-DA (700 g/mol), the Tg increases as the concentration of protein increases in the network. Additionally, MABSA-based bioplastic exhibited lower Tg values than BSA-based bioplastic, which can be attributed to the methacrylate process used to synthesize MABSA, which disturbed the surface charge of the protein. Therefore, MABSA has lower Tg than BSA in this experimental example. The present disclosure describes a tunable system where bioplastic formulations can be chosen based on specific application needs. For example, MABSA 3:1 0.00375% bioplastic is promising for the fabrication of a deployable biomedical device such as a stent, since it has Tg value closest to the human body temperature. In this way, the actuation process will not harm human tissue.

It has been previously demonstrated the shape recovery of BSA-based bioplastics through thermal and hydration stimuli (E. Sanchez-Rexach, et al., ACS Appl. Mater. Interfaces 2021, 13, 19193). In this experimental example, AuNRs were incorporated into the bioplastic systems, which absorb NIR light and transfer the light to heat through surface plasmon resonance, enabling the bioplastic to achieve shape memory under irradiation of NIR laser. FIG. 12D presents the shape memory performance of 3D printed BSA 2:1 bioplastic with AuNRs concentration ranging from 0.0025%-0.005%. The shape memory performance was evaluated in two steps. First, the bioplastic disks were compressed 70% of their original shape for BSA-based and 60% for MABSA-based bioplastic to form a static temporary shape. Then recovery was achieved through irradiation with a NIR laser. Compression was done without raising the temperature above Tg, making the process more convenient and energy saving compared to a traditional shape memory polymer (SMP) that requires programming of temporary structure through heating above Tg (Y. Wang, et al., Adv. Mater. Technol. 2022, 7, 2101058).

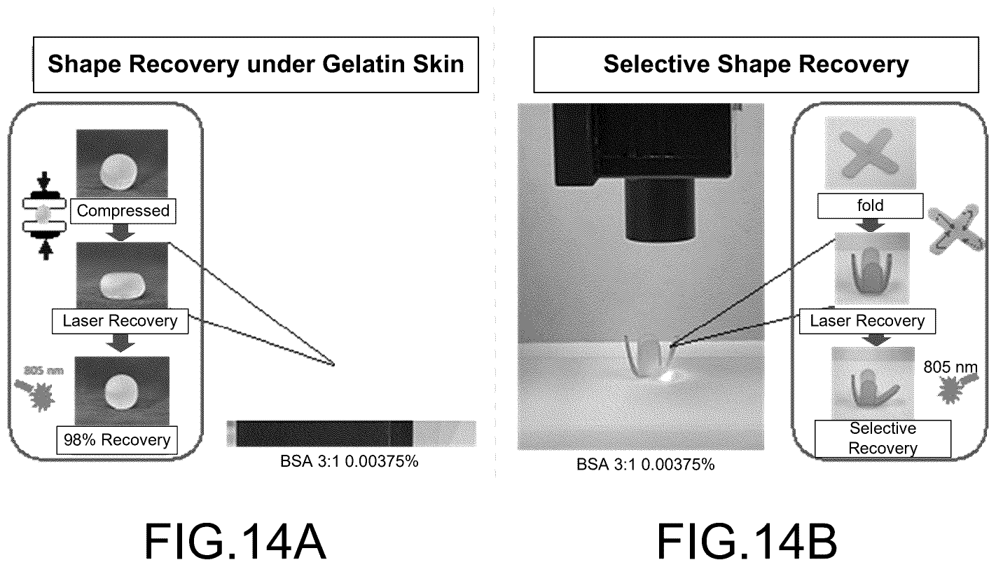

In FIG. 12C, temperature as the function of irradiation time for BSA 2:1 bioplastic with 0-0.005% of AuNRs was plotted. The control sample with 0% of AuNR did not show a noticeable temperature rise after NIR laser irradiation, proving that the protein-polymer network did not absorb the NIR light. Bioplastics containing AuNRs showed a temperature increase upon irradiation, and the higher the concentration of AuNRs, the higher the temperature that can be reached. The recovery process from the compressed structure to the original shape took around 120 s for BSA 2:1 bioplastic in this experimental example. Shape recovery started around 10 seconds of light irradiation, which caused by temperature raised above Tg temperature (FIGS. 12A and 14C). In this example, the recovery percentage for BSA 2:1 printed disk was about 93%-98% after 2 min of irradiation under 3.75 W cm⁻² NIR laser (FIG. 12D).

FIGS. 13A to 15D illustrate compressive stress vs strain curves of example 3D printed bioplastics using BSA 3:1 bioplastic with 0-0.005% AuNRs (FIG. 13A), MABSA 3:1 bioplastic with 0-0.005% AuNRs (FIG. 13B), control samples (0% AuNRs) of 4 different formulated bioplastics (FIG. 13C), and BSA 3:1 0.00375% bioplastic compared with its laser-recovered bioplastic (FIG. 13D).

Uniaxial compression tests were conducted to examine the mechanical properties of bioplastics formulated with BSA and MABSA with varying concentrations of AuNRs, as shown in FIGS. 13A and 15B. Results indicated that the compressive modulus remained unchanged upon the incorporation of the gold nanorods. Therefore, to compare the mechanical properties of the four different formulated bioplastics, control samples (0% AuNRs) were used in FIG. 13C. In this experimental example, the compressive modulus for BSA 2:1, BSA 3:1, MABSA 2:1, and MABSA 3:1 was determined to be 132.5*6.2 MPa, 168.0*5.2 MPa, 108.3*6.7 MPa, and 141.2*6.3 MPa, respectively. As the concentration of the protein increases inside the network, the compressive modulus increases due to the increase of protein junction. In FIGS. 13D and 10C, compressive stress vs. strain curves were presented before and after laser recovery. The graphs indicated that the compressive modulus was nearly identical after NIR laser recovery. It has been previously reported that thermal recovery of printed BSA 3:1 bioplastic at 120° C. for 20 mins would cause the unfolding of BSA, leading to the formation of new bands in the beta-sheet region (1615 cm⁻¹). The beta-sheet formation resulted in an increase in elastic modulus, but a decrease in ductility of the bioplastic in this example (E. Sanchez-Rexach, et al., ACS Appl. Mater. Interfaces 2021, 13, 19193; L. Ma, et al., Polym. Chem. 2013, 4, 5425). In this experimental example, the combination of AuNRs and NIR laser was used to convert light to heat. Broadening of the band was observed at 1648 cm¹ in the amide I region for the compressed bioplastic and no change for laser-recovered bioplastic in this example.

FIG. 14A illustrates an example of shape recovery of BSA 3:1 0.00375% 3D printed bioplastic ball under pork gelatin skin. FIG. 14B illustrates an example of selective shape recovery of a folded 3D-printed BSA 3:1 four-arm flower.

The photothermal performance of 3D-printed bioplastic was further investigated by carrying out a shape recovery experiment under pork gelatin skin, as shown in FIG. 14A. The compressed 3D-printed ball was placed about 1 cm underneath the gelatin skin and exposed to 808 nm NIR laser with a power of 3.75 W cm⁻². The gelatin skin was placed on a FDM printed desk which has a hole in the middle (FIG. 11). After 1 min of NIR laser irradiation, 98% of shape recovery was achieved in this experimental example. Local and selective shape recovery can be demonstrated in a 3D-printed four petal flower, where each petal (11 mm long, 6 mm wide, 1 mm thick) is folded 90 degrees uphold as the temporary structure, as shown in FIG. 14B. A selected petal can be recovered to the original shape by exposing to NIR light with a power of 3.75 W cm-2.

### Conclusions

This experimental example discloses a novel photothermally responsive protein-based shape memory material, based on incorporation of gold nanorods into protein-polymer matrix. The mechanically deformed 3D printed objects were irradiated by light with wavelength corresponding to a specific surface plasmon resonance of gold nanorods, to perform the photothermal shape recovery process. The recovery process was triggered when heat generated by AuNRs exceeded the glass transition temperature of the system. The glass transition temperature of the bioplastic is tunable by changing the protein to polymer ratio. Both BSA and MABSA based bioplastics achieved above 90% shape recovery ratio in 2 mins under irradiation of the NIR-laser with 3.75 W cm⁻² in this experimental example. Mechanical properties remained same after photothermal shape recovery in a subsequent cycle. Selective recovery was achieved with controlled localized irradiation. Shape recovery under the skin was demonstrated using artificial skin made by pork gelatin. 98% of shape recovery was achieved with 1 min of irradiation of 3.75 W cm⁻² NIR laser without drying or damaging the gelatin skin in this experimental example.

### EXAMPLE CLAUSES

The following Example Clauses recite various implementations of the present disclosure. However, implementations of the present disclosure are not necessarily limited to any of the Example Clauses provided herein.
1. A method, including: generating a resin including about 25% to about 30% bovine serum albumin (BSA) and/or methacrylated BSA (MABSA) by weight, about 10% to about 15% a water-soluble co-monomer by weight, about 0.001% to about 0.002% gold nanoparticles by weight, water, and a photoinitiator; generating a hydrogel by exposing the resin to UV-visible light, thereby polymerizing the BSA and/or MABSA and the water-soluble co-monomer; generating a bioplastic in a first shape by removing at least a portion of the water from the hydrogel; converting the bioplastic from the first shape to a second shape by applying a force to the bioplastic; and reverting the bioplastic from the second shape to the first shape by exposing the bioplastic to near infrared (NIR) light.
2. The method of clause 1, wherein the water-soluble co-monomer includes poly(ethylene glycol diacrylate (PEGDA).
3. The method of clause 1 or 2, wherein the gold nanoparticles include nanorods having a length of about 60 nm and a width of about 15 nm.
4. The method of one of clauses 1 to 3, wherein the photoinitiator includes lithium phenyl-2,4,6-trimethylbenzoylphosphinate (LAP).
5. The method of one of clauses 1 to 4, wherein generating the hydrogel by exposing the resin to UV-visible light includes three-dimensionally (3D) printing the hydrogel using a stereolithographic apparatus (SLA) printer.
6. The method of one of clauses 1 to 5, wherein converting the bioplastic from the first shape to the second shape by applying the force to the bioplastic includes compressing the bioplastic.
7. The method of one of clauses 1 to 6, wherein a width of the second shape is shorter than a width of the first shape.
8. The method of one of clauses 1 to 7, wherein a width of the second shape is a percentage of a width of the first shape, the percentage being in a range of about 60% to about 70%.
9. The method of one of clauses 1 to 8, wherein exposing the bioplastic to the NIR light includes converting, by the gold nanoparticles, energy from the NIR light into heat.
10. The method of one of clauses 1 to 9, wherein exposing the bioplastic to the NIR light includes transmitting, by a light source, the NIR light through a biological tissue.
11. The method of clause 10, wherein the light source includes a laser having a power of about 3.75 W*cm⁻².
12. The method of one of clauses 1 to 11, wherein the NIR light has a wavelength of about 808 nm.
13. The method of one of clauses 1 to 12, wherein a plasmon resonance band of the gold nanoparticles overlaps a wavelength of the NIR light.
14. The method of clause 13, wherein the NIR light includes pulses.
15. The method of one of clauses 1 to 14, further including: in response to converting the bioplastic from the first shape to the second shape, inserting the bioplastic into a subject, wherein reverting the bioplastic from the second shape to the first shape by exposing the bioplastic to NIR light occurs when the bioplastic is disposed inside the subject.
16. The method of one of clauses 1 to 15, wherein removing at least the portion of the water from the hydrogel includes drying the hydrogel.
17. A resin, including: a globular protein; a water-soluble co-monomer; water; and light-to-heat converting nanoparticles.
18. The resin of clause 17, wherein the globular protein includes at least one of a serum albumin, pepsin, hemoglobin, lysozyme, lactoglobulin, pea protein, or soy protein.
19. The resin of clause 18, wherein the serum albumin includes BSA and/or MABSA.
20. The resin of one of clauses 17 to 19, wherein the resin includes about 1% to about 50% the globular protein by might.
21. The resin of one of clauses 17 to 20, wherein the resin includes about 20% to about 40% the globular protein by might.
22. The resin of one of clauses 17 to 21, wherein the resin includes about 25% to about 30% the globular protein by might.
23. The resin of one of clauses 17 to 22, wherein the water-soluble co-monomer includes at least one of PEGDA, hydroxyethylacrylate (HEA), or acrylamide (AAm).
24. The resin of one of clauses 17 to 23, wherein the water-soluble co-monomer has a molecular might of about 700 grams per mole (g/mol).
25. The resin of one of clauses 17 to 24, wherein the resin includes about 1% to about 60% the water-soluble co-monomer by weight.
26. The resin of one of clauses 17 to 25, wherein the resin includes about 5% to about 20% the water-soluble co-monomer by weight.
27. The resin of one of clauses 17 to 26, wherein the resin includes about 10% to about 15% the water-soluble co-monomer by weight.
28. The resin of one of clauses 17 to 27, wherein the light-to-heat converting nanoparticles include at least one of nanorods, nanostars, nanospheres, nanocages, nanoclusters, nanoplates, nanotriangles, or nanoshells.
29. The resin of one of clauses 17 to 28, wherein a length of the light-to-heat converting nanoparticles is about 60 nm and/or a width of the light-to-heat converting nanoparticles is about 15 nm.
30. The resin of one of clauses 17 to 29, wherein the resin includes about 0.001% to about 1% the light-to-heat converting nanoparticles by weight.
31. The resin of one of clauses 17 to 30, wherein a plasmon resonance band of the light-to-heat converting nanoparticles corresponds to light having a wavelength in a range of about 780 nm to about 2500 nm.
32. The resin of one of clauses 17 to 31, wherein the light-to-heat converting nanoparticles are configured to generate heat in response to being exposed to NIR light.
33. The resin of one of clauses 17 to 32, wherein a coating of the light-to-heat converting nanoparticles includes PEG.
34. The resin of one of clauses 17 to 33, wherein the light-to-heat converting nanoparticles include at least one of gold or silver.
35. The resin of one of clauses 17 to 34, further including: a photoinitiator.
36. The resin of clause 35, wherein the photoinitiator includes at least one of: LAP; tris(2,2′-bipyridyl)dichlororuthenium(II) hexahydrate (Ru(bpy)3) and sodium persulfate (SPS); or 2-hydroxy-2-methylpropiophenone.
37. The resin of clause 35 or 36, wherein the photoinitiator is configured to polymerize the globular protein and the co-monomer in response to being exposed to UV-visible light.
38. The resin of one of clauses 17 to 37, wherein the globular protein includes MABSA and the nanoparticles include gold nanorods, wherein the resin includes about 30% the MABSA by weight, and wherein the resin includes about 0.0015% the gold nanorods by weight.
39. The resin of one of clauses 17 to 38, wherein the resin has a viscosity in a range of about 0.25 Pa*s to about 10.0 Pa*s.
40. A bioplastic generated by 3D-printing the resin of clause 17.
41. The bioplastic of clause 40, wherein a surface of the bioplastic includes a hydrophobic coating, the hydrophobic coating including trimethylsilane and/or fluoroalkylsilane.
42. The bioplastic of clause 40 or 41, wherein a glass transition temperature (Tg) of the bioplastic is in a range of about 35 to about 55 degrees C.
43. An implantable device including the bioplastic of one of clauses 40 to 42.
44. A method, including: generating a hydrogel by exposing, to UV-visible light, a resin including a globular protein, a water-soluble co-monomer, gold nanoparticles, water, and a photoinitiator; generating a construct by removing at least a portion of the water from the hydrogel; and converting the construct from a first shape to a second shape by applying a force to the construct.
45. The method of clause 44, wherein generating the construct by exposing, to UV-visible light, the resin is performed by an SLA printer.
46. The method of clause 44 or 45, wherein the resin includes about 20% to about 40% the globular protein by weight, and wherein the globular protein includes at least one of BSA, MABSA, pepsin, hemoglobin, lysozyme, lactoglobulin, pea protein, or soy protein.
47. The method of one of clauses 44 to 46, wherein the resin includes about 5% to about 20% the water-soluble co-monomer by weight, and wherein the water-soluble co-monomer includes at least one of PEGDA, HEA, or AAm.
48. The method of one of clauses 44 to 47, wherein the gold nanoparticles include gold nanorods having a length of about 60 nm and/or a width of about 15 nm.
49. The method of one of clauses 44 to 48, wherein the resin includes about 0.001% to about 1% the gold nanoparticles by weight.
50. The method of one of clauses 44 to 49, wherein the photoinitiator includes at least one of: LAP; Ru(bpy)3 and SPS; or 2-hydroxy-2-methylpropiophenone.
51. The method of one of clauses 44 to 50, wherein applying the force to the construct includes at least one of bending the construct, twisting the construct, or compressing the construct.
52. The method of one of clauses 44 to 51, wherein a width of the second shape is shorter than a width of the first shape.
53. The method of one of clauses 44 to 52, wherein a width of the second shape is a percentage of a width of the first shape, the percentage being in a range of about 60% to about 70%.
54. The method of one of clauses 44 to 53, wherein removing at least the portion of the water from the construct includes drying the construct.
55. A shape-restoring bioplastic, including: about 1% to about 95% BSA and/or MABSA by weight; about 25% to about 35% PEGDA by might, the PEGDA being polymerized with the BSA and/or MABSA; and about 0.0025% to about 0.005% gold nanorods.
56. The shape-restoring bioplastic of clause 55, including about 65% to about 75% BSA and/or MABSA by might.
57. The shape-restoring bioplastic of clause 55 or 56, wherein a Tg of the shape-restoring bioplastic is in a range of about 30 to about 55 degrees Celsius.
58. The shape-restoring bioplastic of one of clauses 55 to 57, wherein a ratio of BSA and/or MABSA to PEGDA in the shape-restoring bioplastic is in a range of 2:1 to 3:1 by weight.
59. The shape-restoring bioplastic of one of clauses 55 to 58, wherein a first portion of the shape-restoring bioplastic includes a first ratio of BSA and/or MABSA to PEGDA, and wherein a second portion of the shape-restoring bioplastic includes a second ratio of BSA and/or MABSA to PEGDA, the second ratio being different than the first ratio.
60. The shape-restoring bioplastic of one of clauses 55 to 59, wherein the gold nanorods include first nanorods having a first length and second nanorods having a second length, the second length being different than the first length, wherein a first portion of the shape-restoring bioplastic includes the first nanorods, and wherein a second portion of the shape-restoring bioplastic includes the second nanorods.
61. A stent including the shape-restoring bioplastic of one of clauses 55 to 60.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the,” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

The present disclosure references various books, articles, printed publications, and other materials. Each one of these materials is incorporated by reference herein in its entirety.

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (lie), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, lie, Phe, Met, and Trp; Group 11 (aliphatic): Gly, Ala, Val, Leu, and lie; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.01); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); lie (−1.8); Tyr (−2.3); Phe (−2.5); Trp (−3.4). It is understood that an amino acid can be substituted for another having a similar hydrophilicity value and still obtain a biologically and/or mechanically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like. As indicated elsewhere, variants of gene sequences can include codon optimized variants, sequence polymorphisms, splice variants, and/or mutations that do not affect the function of an encoded product to a statistically-significant degree.

Variants of the protein sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Bil. 215:403-410 (1990); DNASTAR (DNASTAR, Inc., Madison, Wisconsin); and the FASTA program incorporating the Smith-Waterman algorithm (Pearson, Comput. Methods Genome Res., (Proc. Int. Symp) (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

Unless otherwise indicated, the practice of the present disclosure can employ conventional techniques of chemistry, biochemistry, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

Certain implementations are described herein, including the best mode known to the inventors for carrying out implementations of the disclosure. Of course, variations on these described implementations will become apparent to those of ordinary skill in the art upon reading the foregoing description. The inventor expects skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

1-61. (canceled)

62. A resin, comprising:
a globular protein;
a water-soluble co-monomer;
water; and
light-to-heat converting nanoparticles.

63. The resin of claim 62, wherein the globular protein comprises at least one of a serum albumin, pepsin, hemoglobin, lysozyme, lactoglobulin, pea protein, or soy protein; and/or
wherein the globular protein comprises BSA and/or MABSA.

64. The resin of claim 62, wherein the resin comprises about 1% to about 95% the globular protein by weight;
wherein the resin comprises about 1% to about 60% the water-soluble co-monomer by weight; or
wherein the resin has a viscosity in a range of about 0.25 Pa*s to about 10.0 Pa*s.

65. The resin of claim 62, wherein the water-soluble co-monomer comprises at least one of poly(ethylene glycol diacrylate (PEGDA), hydroxyethylacrylate (HEA), or acrylamide (AAm); and/or
wherein the water-soluble co-monomer has a molecular weight of about 700 grams per mole (g/mol).

66. The resin of claim 62, wherein the light-to-heat converting nanoparticles comprise at least one of nanorods, nanostars, nanospheres, nanocages, nanoclusters, nanoplates, nanotriangles, or nanoshells; and/or
wherein a length of the light-to-heat converting nanoparticles is about 60 nm and/or a width of the light-to-heat converting nanoparticles is about 15 nm.

67. The resin of claim 62, wherein a plasmon resonance band of the light-to-heat converting nanoparticles corresponds to light having a wavelength in a range of about 780 nm to about 2500 nm; and/or
wherein the resin comprises about 0.001% to about 1% the light-to-heat converting nanoparticles by weight.

68. The resin of claim 62, wherein the light-to-heat converting nanoparticles are configured to generate heat in response to being exposed to NIR light;
wherein a plasmon resonance band of the light-to-heat converting nanoparticles overlaps a wavelength of the NIR light; and
wherein the NIR light:
has a wavelength of about 808 nm; and/or
comprises pulses.

69. The resin of claim 62, wherein a coating of the light-to-heat converting nanoparticles comprises PEG; and/or
wherein the light-to-heat converting nanoparticles comprise at least one of gold or silver.

70. The resin of claim 62, further comprising:
a photoinitiator comprising at least one of:
lithium phenyl-2,4,6-trimethylbenzoylphosphinate (LAP);
tris(2,2′-bipyridyl)dichlororuthenium(II) hexahydrate (Ru(bpy)3) and sodium persulfate (SPS); or
2-hydroxy-2-methylpropiophenone,
wherein the photoinitiator is configured to polymerize the globular protein and the co-monomer in response to being exposed to UV-visible light.

71. A bioplastic generated by three-dimensionally (3D)-printing a resin comprising:
a globular protein;
a water-soluble co-monomer;
water; and
light-to-heat converting nanoparticles.

72. The bioplastic of claim 71, wherein a surface of the bioplastic comprises a hydrophobic coating, the hydrophobic coating comprising trimethylsilane and/or fluoroalkylsilane;
wherein a glass transition temperature (Tg) of the bioplastic is in a range of about 35 to about 55 degrees C.; or
wherein a ratio of the globular protein to the co-monomer in the bioplastic is in a range of 2:1 to 3:1 by weight.

73. The bioplastic of claim 71, wherein a first portion of the bioplastic comprises a first ratio of the globular protein to the co-monomer, and
wherein a second portion of the bioplastic comprises a second ratio of the globular protein to the co-monomer, the second ratio being different than the first ratio.

74. The bioplastic of claim 71, wherein the light-to-heat converting nanoparticles comprise first gold nanorods having a first length and second gold nanorods having a second length, the second length being different than the first length,
wherein a first portion of the bioplastic comprises the first gold nanorods, and
wherein a second portion of the bioplastic comprises the second gold nanorods.

75. An implantable device comprising the bioplastic of claim 71.

76. A method, comprising:
generating a hydrogel by exposing, to UV-visible light, a resin comprising a globular protein, a water-soluble co-monomer, gold nanoparticles, water, and a photoinitiator;
generating a construct by removing at least a portion of the water from the hydrogel; and
converting the construct from a first shape to a second shape by applying a force to the construct.

77. The method of claim 76, wherein generating the construct by exposing, to UV-visible light, the resin is performed by a stereolithographic apparatus (SLA) printer;
wherein applying the force to the construct comprises at least one of bending the construct, twisting the construct, or compressing the construct; or
wherein removing at least the portion of the water from the construct comprises drying the construct.

78. The method of claim 76, wherein a width of the second shape is shorter than a width of the first shape; and/or
wherein a width of the second shape is a percentage of a width of the first shape, the percentage being in a range of about 60% to about 70%.

79. The method of claim 76, further comprising:
reverting the construct from the second shape to the first shape by exposing the construct to near infrared (NIR) light.

80. The method of claim 79, wherein exposing the construct to the NIR light comprises transmitting, by a light source, the NIR light through a biological tissue, the light source comprising a laser having a power of about 3.75 W*cm².

81. The method of claim 79, further comprising:
in response to converting the construct from the first shape to the second shape, inserting the construct into a subject,
wherein reverting the construct from the second shape to the first shape by exposing the construct to NIR light occurs when the construct is disposed inside the subject.
